(19) United States
(12) Patent Application Publication
(10) Pub. No.: US 2003/0219437 A1
(43) Pub. Date: Nov. 27, 2003

(54) Therapeutic applications of T-BAM (CD40L) technology to treat diseases involving smooth muscle cells

(76) Inventors: Michael J. Yellin, Montclair, NJ (US); Leonard Chess, Scarsdale, NY (US); Mihail N. Karpusas, Roslindale, MA (US); David W. Thomas, Wellesley, MA (US)

Correspondence Address: FISH & NEAVE, 1251 AVENUE OF THE AMERICAS, 50TH FLOOR, NEW YORK, NY, 10020-1105, US

(21) Appl. No.: 10/298,508

(22) Filed: Nov. 15, 2002

Related U.S. Application Data
(63) Continuation of application No. 09/218,523, filed on Dec. 22, 1998, now abandoned.
Application No. 09/218,523 is a continuation of application No. PCT/US97/12925, filed on Jul. 3, 1997.
Application No. PCT/US97/12925 is a continuation of application No. 08/677,730, filed on Jul. 8, 1996, now abandoned.

(51) Int. Cl. A61K 39/395

(52) U.S. Cl. 424/144.1

(57) ABSTRACT

Activation of smooth muscle cells bearing CD40 on their cell surface by CD40 ligand is inhibited by contacting the smooth muscle cells with an anti-T-BAM (CD40L) antibody capable of inhibiting the interactions between CD40 ligand and the CD40-bearing smooth muscle cells, in an amount effective to inhibit activation of the smooth muscle cells. Activation of smooth muscle cells bearing CD40 on their surface by CD40 ligand in a subject is inhibited by administering to the subject an anti-T-BAM (CD40L) antibody capable of inhibiting the interaction between CD40 ligand and the smooth muscle cells, in an amount effective to inhibit activation of the cells. Conditions dependent on CD40 ligand-induced activation of CD40-bearing cells smooth muscle cells are treated, in particular inflammatory bowel disease.

This application claims the priority of U.S. patent application Ser. No. 08/677,730, filed Jul. 8, 1996 the contents of which is hereby incorporated by reference into the present application.

The invention disclosed herein was made with Government support under NIH Grant Nos. K08-AR-01904, RO1-CA55713, RO1-AI-28367, RO1-AI-14969, HL21006, HL42833, HL50629, and RO1-AI-14969 from the Department of Health and Human Services. Accordingly, the U.S. Government has certain rights in this invention.

Throughout this application, various references are referred to within parentheses. Disclosures of these publications in their entireties are hereby incorporated by reference into this application to more fully describe the state of the art to which this invention pertains. Full bibliographic citation for these references may be found in the text or listed by number following the Experimental Details section.

### BACKGROUND OF THE INVENTION

CD40 is a cell surface molecule expressed on a variety of cells and interacts with a 30-33 kDa activation-induced CD4+ T cell counterreceptor termed CD40L. CD40L-CD40 interactions have been extensively studied in T cell-B cell interactions and are essential for T cell dependent B cell differentiation and IgG, IgA and IgE production. CD40 is also expressed on monocytes, dendritic cells, epithelial cells, endothelial cells and fibroblasts. CD40 expression on these cells is upregulated in vitro by cytokines, most notably IFN-γ. Interestingly, in vivo studies have demonstrated markedly upregulated CD40 expression in inflammatory sites, such as rheumatoid arthritis synovial membrane or psoriatic plaques. In vitro studies utilizing anti-CD40 mAb or CD40L+ cells demonstrate that CD40 is functionally expressed on monocytes, dendritic cells, epithelial cells, endothelial cells and fibroblasts.

For example, CD40L-CD40 interactions induce monocytes to secrete the proinflammatory cytokines IL-Iα, IL1β, IL-6 and TNF-α and dendritic cells to secrete TNF-α. CD40L-CD40 interactions also promote monocytes and dendritic cells to secrete the chemokines IL-8 and MIP1α. Moreover, CD40 ligation enhances IL-1 mediated GM-CSF production by thymic epithelial cells. Additionally, CD40L mediated signals induce monocytes to secrete IL-10 and nitric oxide and augment fibroblast IL-6 production. Fibroblasts also proliferate following CD40L-CD40 interactions. Finally, endothelial cells and fibroblasts upregulate intercellular adhesion molecules following CD40 ligation.

Vascular diseases such as atherosclerosis have been treated with a variety of drugs, including cholesterol-lowering drugs, beta blockers, calcium channel blockers, and anti-coagulants. It is now demonstrated that smooth muscle cells are competent to express CD40. This provides a basis for treatment of vascular diseases by inhibition of interactions between CD40 and CD40 ligand (also known as T-BAM, 5c8 Ag, gp39, and TRAP). Other diseases involving smooth muscle are also treated by inhibiting CD40-CD40L interactions.

### SUMMARY OF THE INVENTION

This invention provides a method of inhibiting activation by CD40 ligand of smooth muscle cells bearing CD40 on the surface of the cells, comprising contacting the cells with an agent capable of inhibiting interaction between CD40 ligand and CD40 on the cells, the agent being present in an amount effective to inhibit activation of the cells.

This invention provides a method of inhibiting activation by CD40 ligand of smooth muscle cells bearing CD40 on the surface of the cells, in a subject, comprising administering to the subject an agent capable of inhibiting interaction between CD40 ligand and CD40 on the cells, the agent being present in an amount effective to inhibit activation of the cells in the subject.

This invention provides a method of treating, in a subject, a smooth muscle cell-dependent disease, comprising administering to the subject an agent capable of inhibiting interaction between CD40 ligand and CD40 on the cells, the agent being present in an amount effective to inhibit activation of the cells in the subject and thereby treat the smooth muscle cell-dependent disease.

### DESCRIPTION OF THE FIGURES

FIG. 1A: FACS analysis of resting human aortic smooth muscle cells. The dotted line represents isotype control mAb; the dashed line represents anti-CD54 mAb; and the solid line represents anti-CD40 mAb. This figure shows that smooth muscle cells do not constitutively express CD40.

FIG. 1B: FACS analysis of human aortic smooth muscle cells in the presence of IFN-γ (1000 U/cc) after 72 hours in cell culture. This figure shows upregulation of smooth muscle cell CD40 expression in response to IFN-γ.

FIG. 1C: FACS analysis of human aortic smooth muscle cells in the presence of IL-1α (1 ng/cc) after 72 hours in cell culture. No upregulation of smooth muscle cell CD40 expression was observed.

FIG. 1D: FACS analysis of human aortic smooth muscle cells in the presence of or TNF-α (200 U/cc) after 72 hours in cell culture. No upregulation of smooth muscle cell CD40 expression was observed.

FIGS. 2A-Y: Atomic coordinates of crystal structure of soluble extracellular fragment of human CD40L containing residues Gly116-Leu261 (in Brookhaven Protein Data Bank format). (SEQ ID NO: 1).

FIGS. 3A-3B: CD40 is expressed in situ on smooth muscle cells and macrophages in lesions of transplant atherosclerosis. Shown are photomicrographs of two-color immunohistochemistry studies demonstrating CD40 expression (brown staining) on smooth muscle cells (red staining) in FIG. 3A and macrophages (red staining) in FIG. 3B in a patient with transplant related atherosclerosis.

FIGS. 4A-4B: Normal coronary artery from a patient with idiopathic cardiomyopathy stained with hematoxylin and eosin (FIG. 4A) and anti-CD40 mAb (FIG. 4B). FIG. 4A: Note the absence of intimal thickening or inflammatory infiltrate. FIG. 4B: CD40 expression is restricted to endothelial cells lining the vascular lumen. There was no reactivity with an isotype specific control mAb (not shown). (FIG. 4A, FIG. 4B ×25)

FIGS. 5A-5B: Fibroatheromatous plaque in a coronary artery of a patient with ischemic cardiomyopathy stained with hematoxylin and eosin (FIG. 5A) and anti-CD40 mAb (FIG. 5B). FIG. 5A: The fibrous cap overlying the partially calcified atheromatous core contains numerous inflammatory cells (arrows). FIG. 5B: Most of the inflammatory cells in the fibrous cap are strongly CD40+ (arrows). Adjacent intimal smooth muscle cells and endothelial cells are also CD40+. (FIG. 5A, FIG. 5B ×25)

FIGS. 6A-6C: Early intimal lesion rich in foam cells in a patient with transplant associated coronary artery disease (TCAD) stained with hematoxylin and eosin (FIG. 6A) and anti-CD40 mAb (FIG. 6B, FIG. 6C). FIG. 6A: The intimal lesion contains numerous foam cells, macrophages and smooth muscle cells. FIG. 6B: CD40 is strongly expressed on many intimal cells in this early lesion of TCAD. FIG. 6C: In particular, foam cells showed abundant staining for CD40. (FIG. 6A ×25, FIG. 6B ×50, FIG. 6C ×400).

FIGS. 7A-7D: Inflammatory infiltrate present in the fibrous cap of intimal lesion in native CA labelled with anti-CD40L mAb (FIG. 7A), control mAb (FIG. 7B), anti-CD4 mAb (FIG. 7C) and anti-CD8 mAb (FIG. 7D). FIG. 7A: Characteristic cytoplasmic and cell surface CD40L immunoreactivity which was restricted to lymphocytes. FIG. 7B: The same lesion stained with an irrelevant isotype matched control mAb shows no immunostaining. FIG. 7C: Virtually all lymphocytes in native CA lesions (as well as many macrophages and foam cells) were CD4⁺, suggesting that the CD40L⁺ lymphocytes are CD4⁺ T cells. FIG. 7D: CD8⁺ T cells were rare in intimal plaques of native CA. (FIGS. 7A, 7B ×1000, FIGS. 7C, 7D ×400)

FIGS. 8A-8C: Deep intimal lymphoid aggregates in TCAD labelled with anti-CD40L mAb (FIG. 8A), control mAb (FIG. 8B) and anti-CD4 mAb (FIG. 8C). FIG. 8A: Most of the CD40L⁺ cells in TCAD (arrows) were found in lymphoid aggregates within the intima and away from the endothelial surface. FIG. 8B: The irrelevant isotype matched control mAb shows no cellular staining in such intimal lymphoid aggregates. FIG. 8C: The same intimal lymphoid aggregate as above contains almost exclusively CD4⁺ T cells suggesting that CD40L is expressed on CD4⁺ T cells in these lesions. (FIGS. 8A-8C ×400).

FIGS. 9A-9B: Focus of endothelitis in TCAD stained with anti-CD8 (FIG. 9A) and anti-CD40L (FIG. 9B) mAbs. FIG. 9A: CD8⁺ T cells attached to the luminal endothelial cells in TCAD characteristic for endothelitis. Most of the CD8⁺ T cells were present in foci of endothelitis, whereas they were rarely present in intimal lymphoid aggregates away from the endothelial surface. FIG. 9B: Inflammatory cells in foci of endothelitis are CD40L⁻. Similarly, CD40L expression was not detected on endothelial cells. (FIGS. 9A-9B ×400)

FIGS. 10A-10B: FIG. 10A: Double immunolabelling of intimal lesion of native CA with anti-CD40 mAb (brown) and anti-CD68 mAb (red), a marker for macrophages. The central cluster of cells (arrows) shows strong staining for both CD40 and CD68. FIG. 10B: Double immunolabelling of TCAD with anti-CD40 mAb (brown) and anti-smooth muscle actin mAb (red) demonstrates CD40+ smooth muscle cells (arrows). CD40 reactivity is confined to intimal smooth muscle cells (arrows), whereas medial myocytes were CD40−. (FIGS. 10A-B ×400)

FIGS. 11A-11D: Serial sections of native CA demonstrating intimal neovascularization and stained with anti-CD34 (FIG. 11A), anti-CD40 (FIG. 11B), anti-ICAM-1 (FIG. 11C), and anti-VCAM-1 (FIG. 11D) mAbs. FIG. 11A: Endothelial cells of intimal neovessels highlighted by CD34 staining. FIG. 11B: Intimal neovascular endothelial cells strongly express CD40. The adjacent inflammatory cells also label for CD40. FIGS. 11C, 11D: Foci of neovascularization also showed strong endothelial reactivity for ICAM-1 (FIG. 11C), and VCAM-1 (FIG. 11D) (FIGS. 11A-11D ×400).

FIGS. 12A-12C: Double immunolabelling of actively inflamed intimal lesion of native CA with anti-CD40 mAb (brown) and adhesion molecules (red) anti-ICAM-1 mAb (FIG. 12A), anti-VCAM-1 mAb (FIG. 12B) and irrelevant control mAb (FIG. 12C). FIG. 12A: Virtually all CD40⁺ (brown) cells, predominantly macrophages (long arrows), and intimal myocytes (short arrows), are strongly reactive for ICAM-1 (red). FIG. 12B: A large number of CD40⁺ (brown) inflammatory cells and intimal myocytes (arrows) are also reactive for VCAM-1 (red). FIG. 12C: Same intimal lesion double labelled for CD40 (brown) and irrelevant isotype matched control Ab substituted for anti-ICAM-1 and anti-VCAM-1 mAbs (red). Only brown and no red staining is discerned indicating absence of interference of detection techniques for the sequentially applied anti-CD40 and anti-ICAM or anti-VCAM mAbs (see Materials and Methods). (FIGS. 12A-C ×400).

FIG. 13: Double immunolabelling of intimal lesion of native CA with anti-p65 mAb labelling activated NF-kB (brown) and CD40 (red). Activated NF-κB was exclusively discerned in nuclei of CD40⁺ cells (arrows), most of which are macrophages. (×400).

### DETAILED DESCRIPTION

This invention provides a method of inhibiting activation by CD40 ligand of smooth muscle cells bearing CD40 on the cell surface, comprising contacting the cells with an agent capable of inhibiting interaction between CD40 ligand and CD40 on the cells, the agent being present in an amount effective to inhibit activation of the cells. In one embodiment of this invention the agent is capable of inhibiting any interaction between CD40 ligand and CD40. “Interaction between CD40 ligand and CD40 on the cells” refers to one or more aspects, functional or structural, of a CD40-CD40 ligand interrelationship. Therefore, in one embodiment, an agent which inhibits interaction may competitively bind to CD40 ligand in such a way to block or diminish the binding of CD40 ligand to cellular CD40. In another embodiment an agent which inhibits interaction may associate with CD40 or CD40 ligand in a manner which does not inhibit binding of CD40 ligand to cellular CD40, but which influences the cellular response to the CD40 ligation, such as by altering the turnover rate of the cellular CD40 or the CD40-agent complex, by altering binding kinetics of CD40 with CD40 ligand, or by altering the rate or extent of cellular activation in response to CD40 ligation.

In specific embodiments the CD40-bearing smooth muscle cells are smooth muscle cells of the bladder, vascular smooth muscle cells, bronchial smooth muscle cells, aortic smooth muscle cells, coronary smooth muscle cells, pulmonary smooth muscle cells, or gastrointestinal smooth muscle cells. In more specific embodiments the gastrointestinal smooth muscle cells are esophageal, stomach, or intestinal smooth muscle cells, including smooth muscle cells of the small intestine or the large intestine (bowel).

In an embodiment of this invention the agent inhibits binding of CD40 ligand to CD40 on the cells.

In an embodiment of this invention the agent is a protein.

In another embodiment of this invention the agent is a nonprotein. As used herein the term nonprotein includes any and all compounds or agents which encompass elements other than simple or conjugated polypeptide chains. This includes elements such as amino acids having non-peptide linkages; nonprotein amino acids such as β, γ, or δ amino acids, amino acids in D configuration, or other nonprotein amino acids including homocysteine, homoserine, citrulline, ornithine, γ-aminobutyric acid, canavanine, djenkolic acid, or β-cyanoalanine; monosaccharides, polysaccharides, or carbohydrate moieties; fatty acids or lipid moieties; nucleotide moieties, mineral moieties; or other nonprotein elements.

In another embodiment of this invention, the agent is a peptidomimetic compound. The peptidomimetic compound may be at least partially unnatural. The peptidomimetic compound may be a small molecule mimic. The compound may have increased stability, efficacy, potency and bioavailability by virtue of the mimic. Further, the compound may have decreased toxicity. The peptidomimetic compound may have enhanced mucosal intestinal permeability. The compound may be synthetically prepared. The compound of the present invention may include L-,D- or unnatural amino acids, alpha, alpha-disubstituted amino acids, N-alkyl amino acids, lactic acid (an isoelectronic analog of alanine). The peptide backbone of the compound may have at least one bond replaced with PSI-[CH═CH] (Kempf et al. (1991) Intl. J. Peptide and Prot. Res. 38, 237-241). The compound may further include trifluorotyrosine, p-Cl-phenylalanine, p-Br-phenylalanine, poly-L-propargylglycine, poly-D,L-allyl glycine, or poly-L-allyl glycine.

In another embodiment of the present invention, the peptidomimetic compound having the biological activity of inhibiting interaction between CD40 ligand and CD40 on cells may have a bond, a peptide backbone or an amino acid component replaced with a suitable mimic. Examples of unnatural amino acids which may be suitable amino acid mimics include β-alanine, L-α-amino butyric acid, L-γ-amino butyric acid, L-α-amino isobutyric acid, L-ε-amino caproic acid, 7-amino heptanoic acid, L-aspartic acid, L-glutamic acid, cysteine (acetamindomethyl), N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, Boc-L-thioproline. (Blondelle, S. E. et al., (1994) Antimicrobial Agents and Chemotherapy 38, 2280-2286.; Pinilla, C., et al. (1995) Peptide Science 37, 221-240).

In a specific embodiment the protein comprises an antibody or portion thereof capable of inhibiting interaction between CD40 ligand and CD40 on the cells. The antibody is a monoclonal or polyclonal antibody. In a more specific embodiment the monoclonal antibody specifically binds to the epitope to which monoclonal antibody 5c8 (ATCC Accession No. HB 10916) specifically binds. An example of such a monoclonal antibody is monoclonal antibody 5c8 (ATCC Accession No. HB 10916). In another embodiment, the antibody specifically binds to CD40. One example of an anti-CD40 antibody is the monoclonal mouse anti-human CD40, available from Genzyme Customer Service (Product 80-3702-01, Cambridge, Mass.). In other embodiments the monoclonal antibody is a chimeric antibody, a primatized antibody, a humanized antibody, or an antibody which includes a CDR region from a first human and an antibody scaffold from a second human.

The meaning of “chimeric”, “primatized” and “humanized” antibody and methods of producing them are well known to those of skill in the art. See, for example, PCT International Publication No. WO 90/07861, published Jul. 26, 1990 (Queen, et al.); and Queen, et al. Proc. Nat'l Acad. Sci.-USA (1989) 86: 10029. Methods of making primatized antibodies are disclosed, for example, in PCT International publication No. WO __/02108, corresponding to International Application No. PCT/US92/06194 (Idec Pharmaceuticals); and in Newman, et al., Biotechnology (1992) 10:1455-1460, which are hereby incorporated by reference into this application.

Generally, a humanized antibody is an antibody comprising one or more complementarity determining regions (CDRs) of a non-human antibody functionally joined to human framework region segments. Additional residues associated with the non-human antibody can optionally be present. Typically, at least one heavy chain or one light chain comprises non-human CDRs. Typically, the non-human CDRs are mouse CDRs. Generally, a primatized antibody is an antibody comprising one or more complementarity determining regions (CDRs) of an antibody of a species other than a non-human primate, functionally joined to framework region segments of a non-human primate. Additional residues associated with the species from which the CDR is derived can optionally be present. Typically, at least one heavy chain or one light chain comprises CDRs of the species which is not a nonhuman primate. Typically, the CDRs are human CDRs. Generally, a chimeric antibody is an antibody whose light and/or heavy chains contain regions from different species. For example one or more variable (V) region segments of one species may be joined to one or more constant (C) region segments of another species. Typically, a chimeric antibody contains variable region segments of a mouse joined to human constant region segments, although other mammalian species may be used.

Monoclonal antibody 5c8 is produced by a hybridoma cell which was deposited on Nov. 14, 1991 with the American Type Culture Collection (ATCC), 12301 Parklawn Drive, Rockville, Md. 20852, U.S.A. under the provisions of the Budapest Treaty for the International Recognition of the Deposit of Microorganisms for the Purposes of Patent Procedure. The hybridoma was accorded ATCC Accession Number HB 10916.

In a specific embodiment the portion of the antibody comprises a complementarity determining region or variable region of a light or heavy chain. In another specific embodiment the portion of the antibody comprises a complementarity determining region or a variable region. In another specific embodiment the portion of the antibody comprises a Fab or a single chain antibody. A single chain antibody is made up of variable regions linked by protein spacers in a single protein chain.

In another embodiment the protein comprises soluble extracellular region of CD40 ligand, or portion thereof, or variant thereof, capable of inhibiting interaction between CD40 ligand and CD40 on the cells; or soluble extracellular region of CD40, or portion thereof, or variant thereof, capable of inhibiting interaction between CD40 ligand and CD40 on the cells. In a specific embodiment the soluble extracellular region of CD40 ligand or CD40 is a monomer. In another embodiment the soluble extracellular region of CD40 is an oligomer.

Variants can differ from naturally occurring CD40 or CD40 ligand in amino acid sequence or in ways that do not involve sequence, or both. Variants in amino acid sequence are produced when one or more amino acids in naturally occurring CD40 or CD40 ligand is substituted with a different natural amino acid, an amino acid derivative or non-native amino acid. Particularly preferred variants include naturally occurring CD40 or CD40 ligand, or biologically active fragments of naturally occurring CD40 or CD40 ligand, whose sequences differ from the wild type sequence by one or more conservative amino acid substitutions, which typically have minimal influence on the secondary structure and hydrophobic nature of the protein or peptide. Variants may also have sequences which differ by one or more non-conservative amino acid substitutions, deletions or insertions which do not abolish the CD40 or CD40 ligand biological activity. Conservative substitutions typically include the substitution of one amino acid for another with similar characteristics such as substitutions within the following groups: valine, glycine; glycine, alanine; valine, isoleucine; aspartic acid,glutamic acid; asparagine,glutamine; serine,threonine; lysine, arginine; and phenylalanine,tyrosine. The non-polar (hydrophobic) amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid.

Other conservative substitutions can be taken from Table 1, and yet others are described by Dayhoff in the Atlas of Protein Sequence and Structure (1988).

TABLE 1
Conservative Amino Acid Replacements

| For Amino Acid | Code | Replace with any of |
|---|---|---|
| Alanine | A | D-Ala, Gly, beta-ALa, L-Cys, D- |
| | | Cys |
| Arginine | R | D-Arg, Lys, homo-Arg, D-homo- |
| | | Arg, Met, D-Met, Ile, D-Ile, |
| | | Orn, D-Orn |
| Asparagine | N | D-Asn, Asp, D-Asp, Glu, D-Glu, |
| | | Gln, D-Gln |
| Aspartic Acid | D | D-Asp, D-Asn, Asn, Glu, D-Glu, |
| | | Gln, D-Gln |
| Cysteine | C | D-Cys, S-Me-Cys, Met, D-Met, Thr, |
| | | D-Thr |
| Glutamine | Q | D-Gln, Asn, D-Asn, Glu, D-Glu, Asp, |
| | | D-Asp |
| Glutamic Acid | E | D-Glu, D-Asp, Asp, Asn, D-Asn, |
| | | Gln, D-Gln |
| Glycine | G | Ala, D-Ala, Pro, D-Pro, Beta- |
| | | Ala, Acp |
| Isoleucine | I | D-Ile, Val, D-Val, Leu, D-Leu, |
| | | Met, D-Met |
| Leucine | L | D-Leu, Val, D-Val, Met, D-Met |
| Lysine | K | D-Lys, Arg, D-Arg, homo-Arg, D- |
| | | homo-Arg, Met, D-Met, Ile, D- |
| | | Ile, Orn, D-Orn |
| Methionine | M | D-Met, S-Me-Cys, Ile, D-Ile, |
| | | Leu, D-Leu, Val, D-Val, Norleu |
| Phenylalanine | F | D-Phe, Tyr, D-Thr, L-Dopa, His, D- |
| | | His, Trp, D-Trp, Trans 3, 4 or |
| | | 5-phenylproline, cis 3, 4 or 5 |
| | | phenylproline |
| Proline | P | D-Pro, L-I-thioazolidine-4- |
| | | carboxylic acid, D- or L-1- |
| | | oxazolidine-4-carboxylic acid |
| Serine | S | D-Ser, Thr, D-Thr, allo-Thr, |
| | | Met, D-Met, Met(O), D-Met(O), |
| | | Val, D-Val |
| Threonine | T | D-Thr, Ser, D-Ser, allo-Thr, |
| | | Met, D-Met, Met(O) D-Met(O), |
| | | Val, D-Val |
| Tyrosine | Y | D-Tyr, Phe, D-Phe, L-Dopa, |
| | | His, D-His |
| Valine | V | D-Val, Leu, D-Leu, Ile, D-Ile, |
| | | Met, D-Met |

Other variants within the invention are those with modifications which increase peptide stability. Such variants may contain, for example, one or more non-peptide bonds (which replace the peptide bonds) in the peptide sequence. Also included are: variants that include residues other than naturally occurring L-amino acids, such as D-amino acids or non-naturally occurring or synthetic amino acids such as beta or gamma amino acids and cyclic variants. Incorporation of D- instead of L-amino acids into the polypeptide may increase its resistance to proteases. See, e.g., U.S. Pat. No. 5,219,990.

The peptides of this invention may also be modified by various changes such as insertions, deletions and substitutions, either conservative or nonconservative where such changes might provide for certain advantages in their use.

In other embodiments, variants with amino acid substitutions which are less conservative may also result in desired derivatives, e.g., by causing changes in charge, conformation and other biological properties. Such substitutions would include for example, substitution of hydrophilic residue for a hydrophobic residue, substitution of a cysteine or proline for another residue, substitution of a residue having a small side chain for a residue having a bulky side chain or substitution of a residue having a net positive charge for a residue having a net negative charge. When the result of a given substitution cannot be predicted with certainty, the derivatives may be readily assayed according to the methods disclosed herein to determine the presence or absence of the desired characteristics.

Variants within the scope of the invention include proteins and peptides with amino acid sequences having at least eighty percent homology with the extracellular region of CD40 or the extracellular region of CD40 ligand. More preferably the sequence homology is at least ninety percent, or at least ninety-five percent.

Just as it is possible to replace substituents of the scaffold, it is also possible to substitute functional groups which decorate the scaffold with groups characterized by similar features. These substitutions will initially be conservative, i.e., the replacement group will have approximately the same size, shape, hydrophobicity and charge as the original group. Non-sequence modifications may include, for example, in vivo or in vitro chemical derivatization of portions of naturally occurring CD40 or CD40 ligand, as well as changes in acetylation, methylation, phosphorylation, carboxylation or glycolsylation.

In a further embodiment the protein, including the extracellular region of CD40 ligand and CD40, is modified by chemical modifications in which activity is preserved. For example, the proteins may be amidated, sulfated, singly or multiply halogenated, alkylated, carboxylated, or phosphorylated. The protein may also be singly or multiply acylated, such as with an acetyl group, with a farnesyl moiety, or with a fatty acid, which may be saturated, monounsaturated or polyunsaturated. The fatty acid may also be singly or multiply fluorinated. The invention also includes methionine analogs of the protein, for example the methionine sulfone and methionine sulfoxide analogs. The invention also includes salts of the proteins, such as ammonium salts, including alkyl or aryl ammonium salts, sulfate, hydrogen sulfate, phosphate, hydrogen phosphate, dihydrogen phosphate, thiosulfate, carbonate, bicarbonate, benzoate, sulfonate, thiosulfonate, mesylate, ethyl sulfonate and benzensulfonate salts.

The soluble, monomeric CD40-L protein can comprise all or part of the extracellular region of CD40-L. The extracellular region of CD40-L contains the domain that binds to CD40. Thus, soluble CD40-L can inhibit the interaction between CD40L and the CD40-bearing cell. This invention contemplates that sCD40-L may constitute the entire extracellular region of CD40-L, or a fragment or derivative containing the domain that binds to CD40.

Soluble CD40 protein (sCD40) comprises the extracellular region of CD40. sCD40 inhibits the interaction between CD40L and CD40-bearing cells. sCD40 may be in monomeric or oligomeric form.

In another embodiment of this invention the protein comprising soluble extracellular region of CD40 or portion thereof further comprises an Fc region fused to the extracellular region of CD40 or portion thereof. In a specific embodiment the Fc region is capable of binding to protein A or protein G. In another embodiment the Fc region comprises IgG, IgG₁, IgG₂, IgG₃, IgG₄, IgA, IgA₁, IgA₂, IgM, IgD, or IgE.

The soluble CD40/Fc fusion protein can be prepared using conventional techniques of enzymes cutting and ligation of fragments from desired sequences. Suitable Fc regions for the fusion protein are Fc regions that can bind to protein A or protein G, or that are capable of recognition by an antibody that can be used in purification or detection of a fusion protein comprising the Fc region. For example, the Fc region may include the Fc region of human IgG₁ or murine IgG₁. This invention also provides a nucleic acid molecule which encodes the CD40/Fc fusion protein.

The method of creating soluble forms of membrane molecules by recombinant means, in which sequences encoding the transmembrane and cytoplasmic domains are deleted, is well known. See generally Hammonds et al., U.S. Pat. No. 5,057,417. In addition, methods of preparing sCD40 and CD40/Fc fusion protein are well-known. See, e.g., PCT International Publication No. WO 93/08207; Fanslow et al., “Soluble Forms of CD40 Inhibit Biologic Responses of Human B Cells,” J. Immunol., vol. 149, pp.655-60 (July 1992).

In an embodiment of this invention, the agent is selected by a screening method.

In a specific embodiment the agent is selected by a screening method, which comprises isolating a sample of cells; culturing the sample under conditions permitting activation of CD40-bearing cells; contacting the sample with cells expressing a protein which is specifically recognized by monoclonal antibody 5c8 produced by the hybridoma having ATCC Accession no. HB 10916, or with a protein which is specifically recognized by monoclonal antibody 5c8 produced by the hybridoma having ATCC Accession no. HB 10916, effective to activate the CD40-bearing cells; contacting the sample with an amount of the agent effective to inhibit activation of the CD40-bearing cells if the agent is capable of inhibiting activation of the CD40-bearing cells; and determining whether the cells expressing the protein which is specifically recognized by monoclonal antibody 5c8 produced by the hybridoma having ATCC Accession no. HB 10916, or with the protein which is specifically recognized by monoclonal antibody 5c8 produced by the hybridoma having ATCC Accession no. HB 10916, activate the CD40-bearing cells in the presence of the agent. The cell sample may be isolated from diverse tissues, including cell lines in culture or cells isolated from an animal, such as dispersed cells from a solid tissue, cells derived from a bone marrow bipsy, or cells isolated from a body fluid such as blood or lymphatic fluid.

In another specific embodiment the agent (molecule) is selected based on a three-dimensional structure of soluble extracellular region of CD40 ligand or portion thereof capable of inhibiting interaction between CD40 ligand and CD40 on the cells. The agent may be selected from a library of known agents, modified from a known agent based on the three-dimensional structure, or designed and synthesized de novo based on the three-dimensional structure. In specific embodiments the agent (molecule) is designed by structure optimization of a lead inhibitory agent based on a three-dimensional structure of a complex of the soluble extracellular region of CD40 ligand or portion thereof with the lead inhibitory agent. A lead inhibitory agent is a molecule which has been identified which, when it is contacted with CD40 ligand, binds to and complexes with the soluble extracellular region of CD40 ligand, CD40, or portion thereof, thereby decreasing the ability of the complexed or bound CD40 ligand or CD40 ligand portion to activate CD40-bearing cells. In another embodiment, a lead inhibitory agent may act by interacting with either the extracellular region of CD40 ligand, CD40, or in a tertiary complex with both a portion of CD40 ligand and CD40, decreasing the ability of the complexed CD40 ligand-CD40 to activate the CD40-bearing cells. In the methods of the invention, the CD40 ligand may be either soluble or bound to cells such as activated T cells, and may be either full length native CD40 ligand or portions thereof. Decreased ability to activate CD40-bearing cells may be measured in different ways. One way it may be measured is by showing that CD40 ligand, in the presence of inhibitor, causes a lesser degree of activation of CD40-bearing cells, as compared to treatment of the cells with a similar amount of CD40 ligand without inhibitor under similar conditions. Decreased ability to activate CD40-bearing cells may also be indicated by a higher concentration of inhibitor-CD40 ligand complex being required to produce a similar degree of activation of CD40-bearing cells under similar conditions, as compared to unbound CD40 ligand. At the extreme, the inhibitor-contacted CD40 ligand may be unable to activate CD40-bearing cells at concentrations and under conditions which allow activation of these cells by unbound CD40 ligand or a given portion thereof.

The agent (molecule) can be selected by a computational screening method using the crystal structure of a soluble fragment of the extracellular domain of human CD40L containing residues Gly116-Leu261 (sCD40L(116-261)).

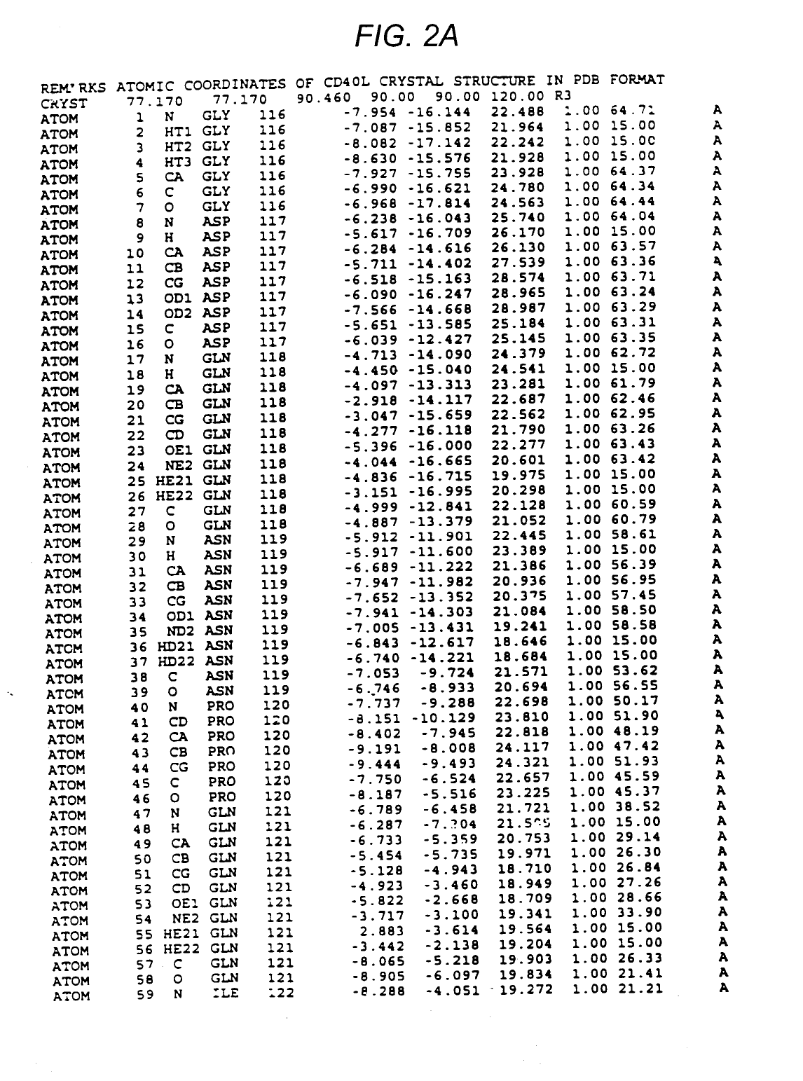

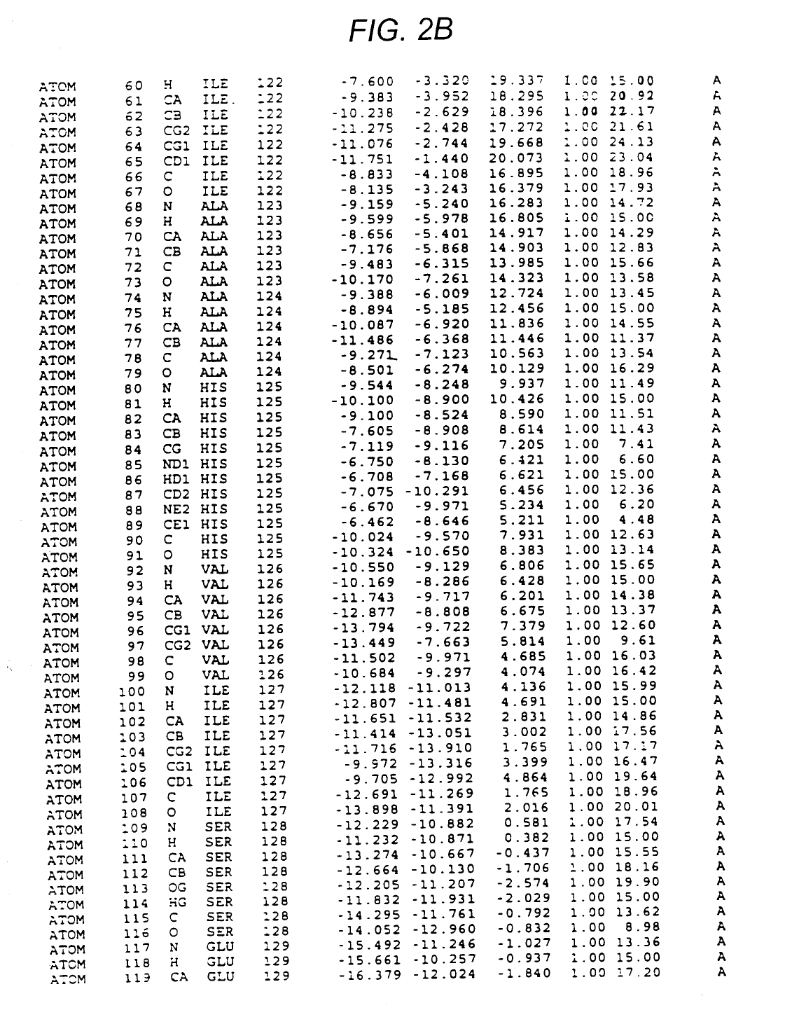

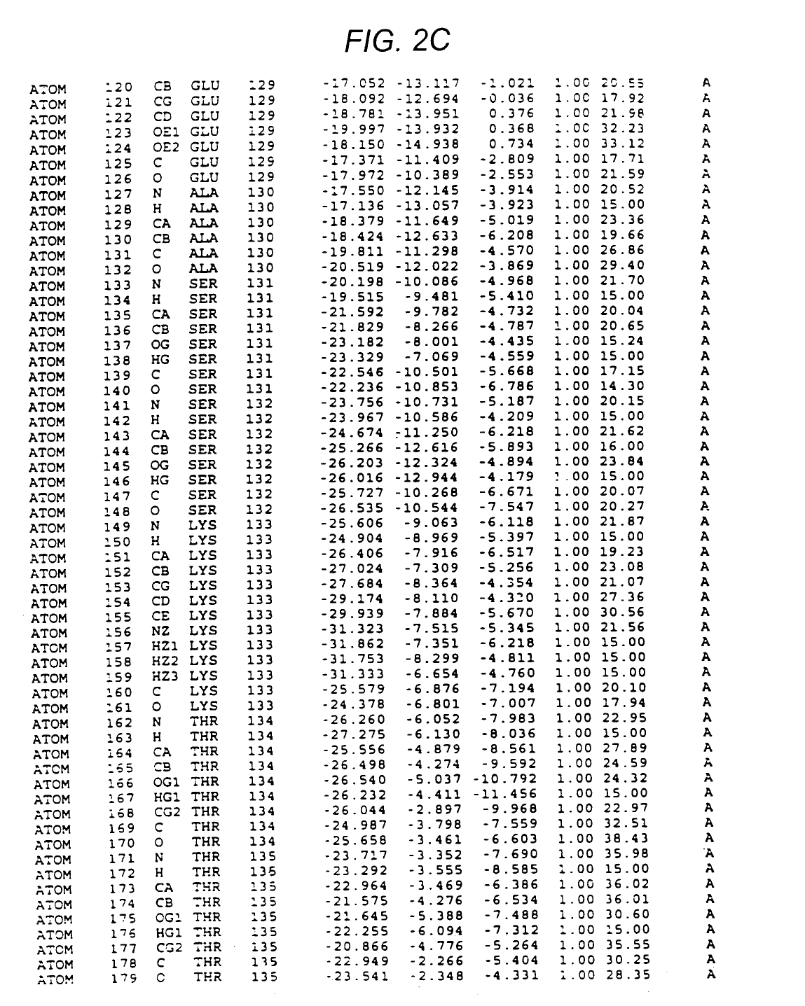

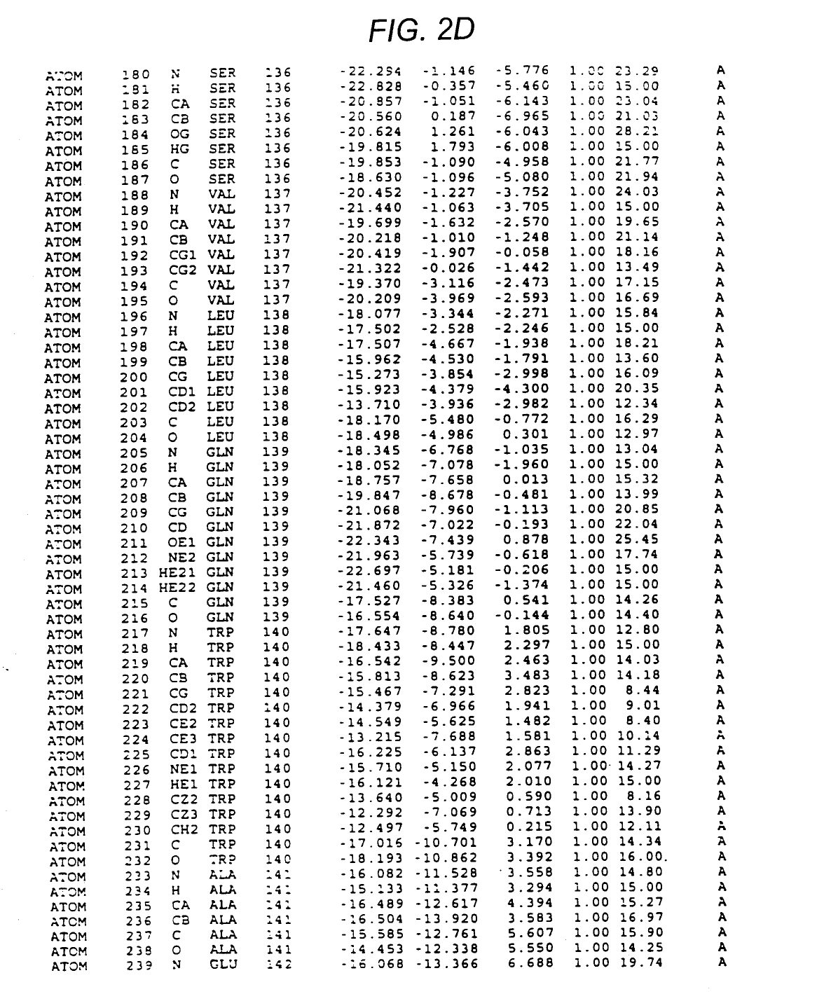

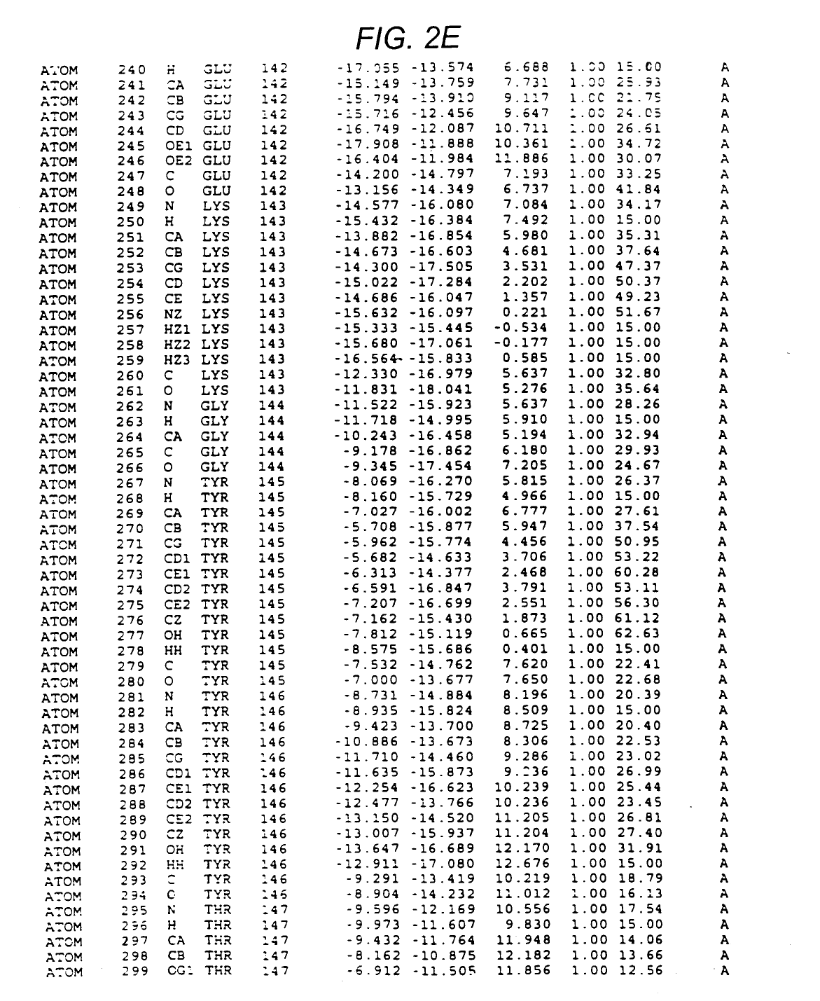

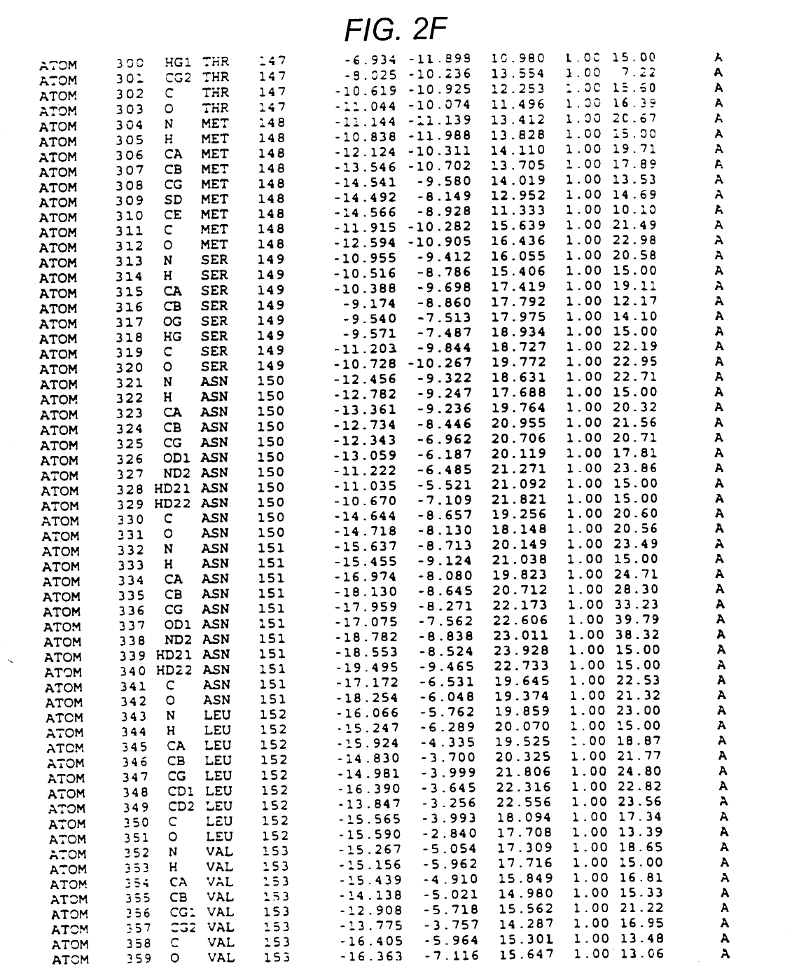

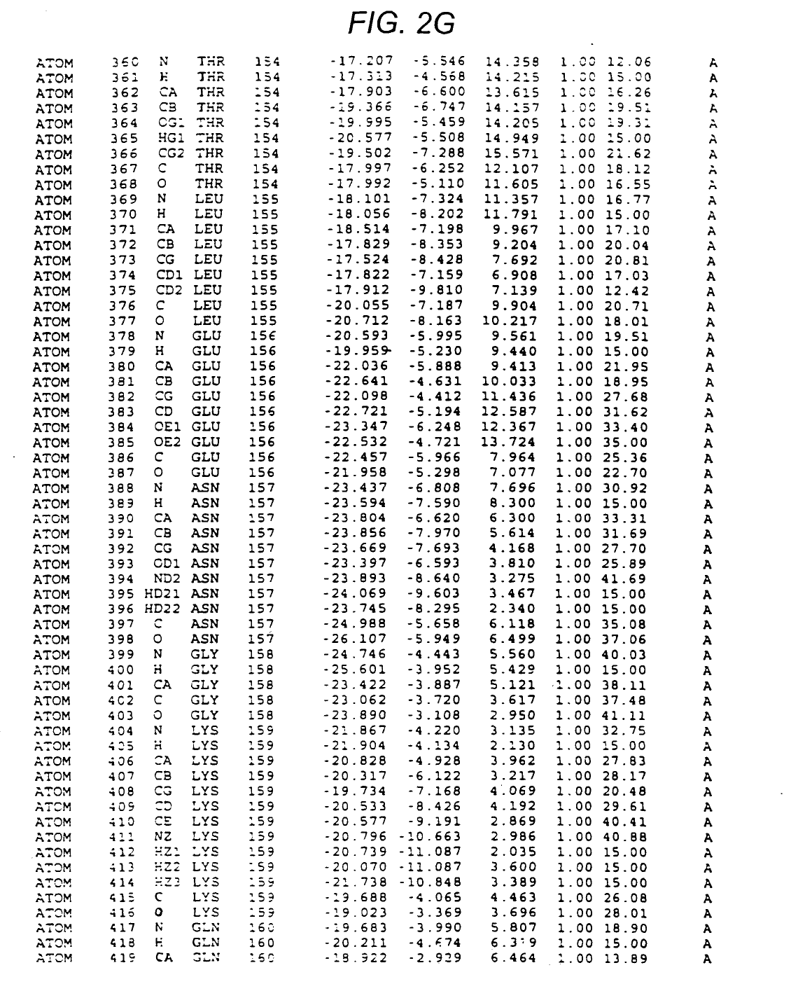

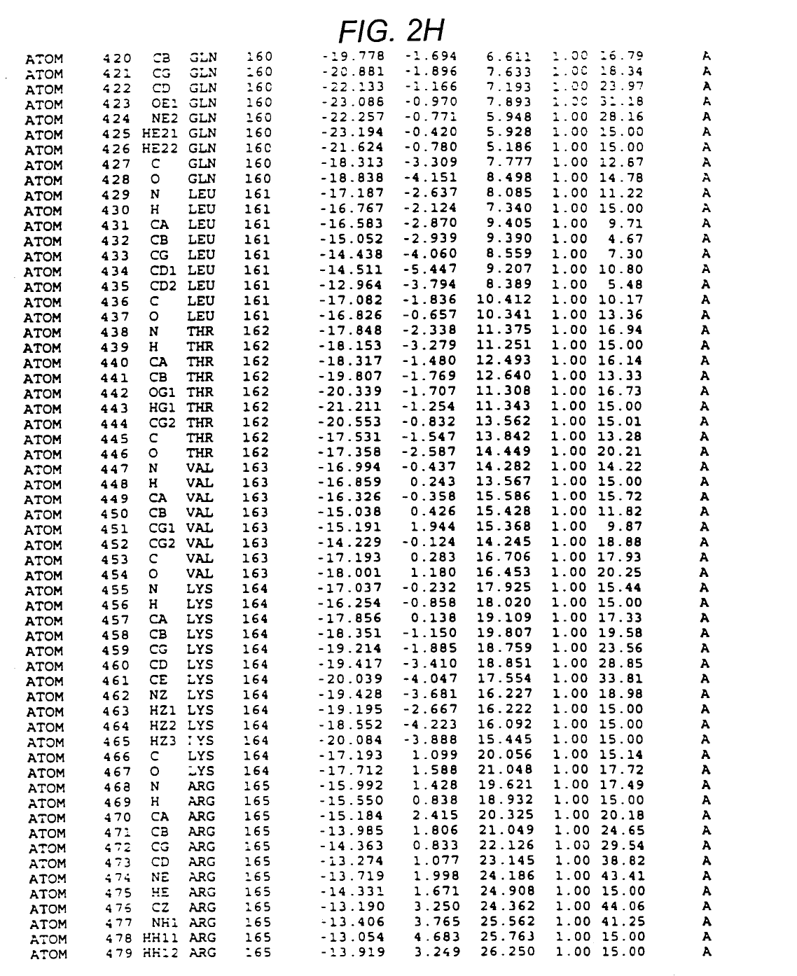

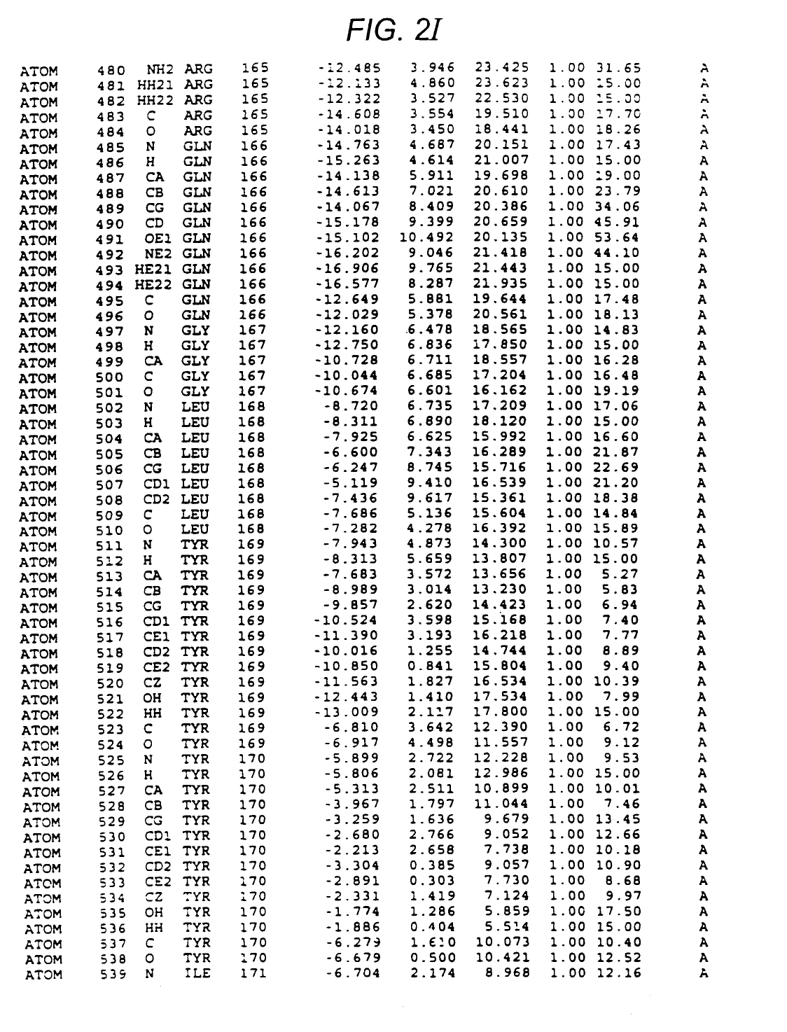

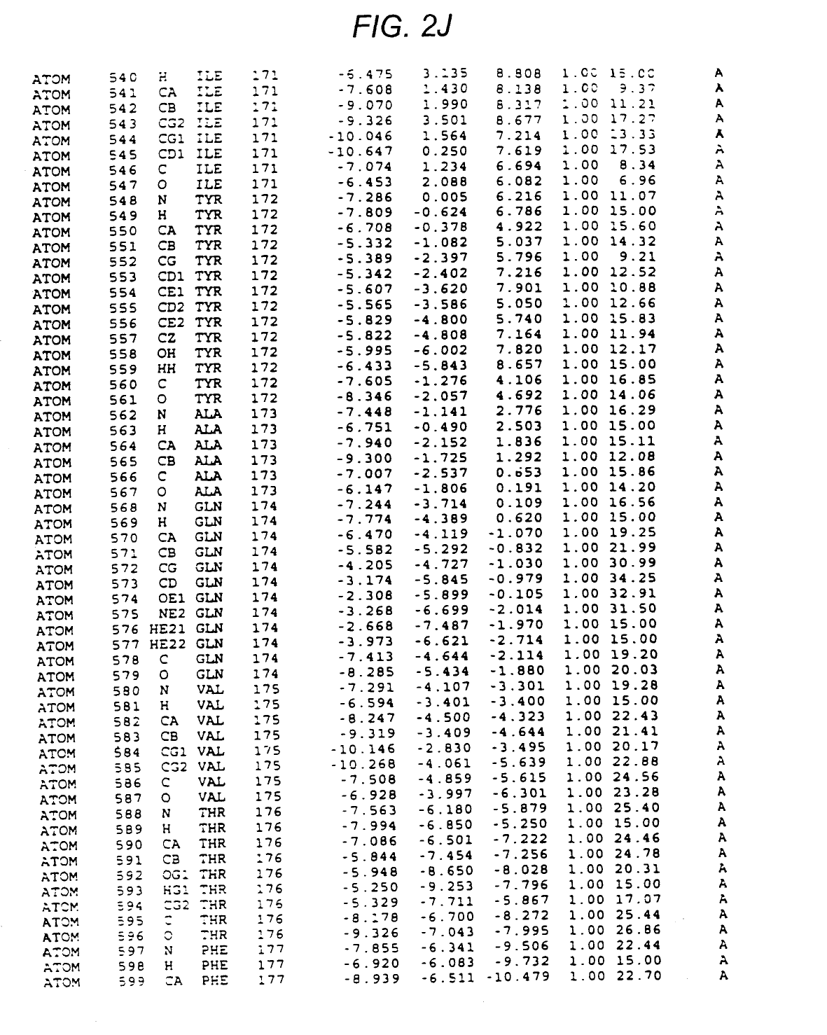

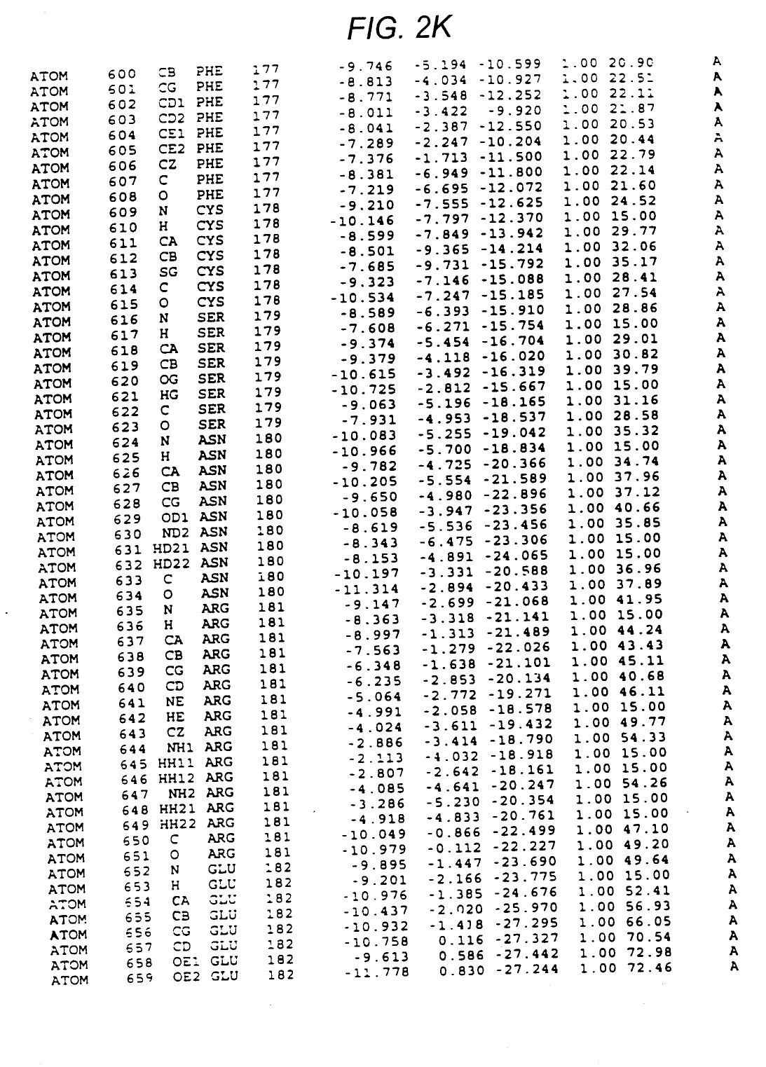

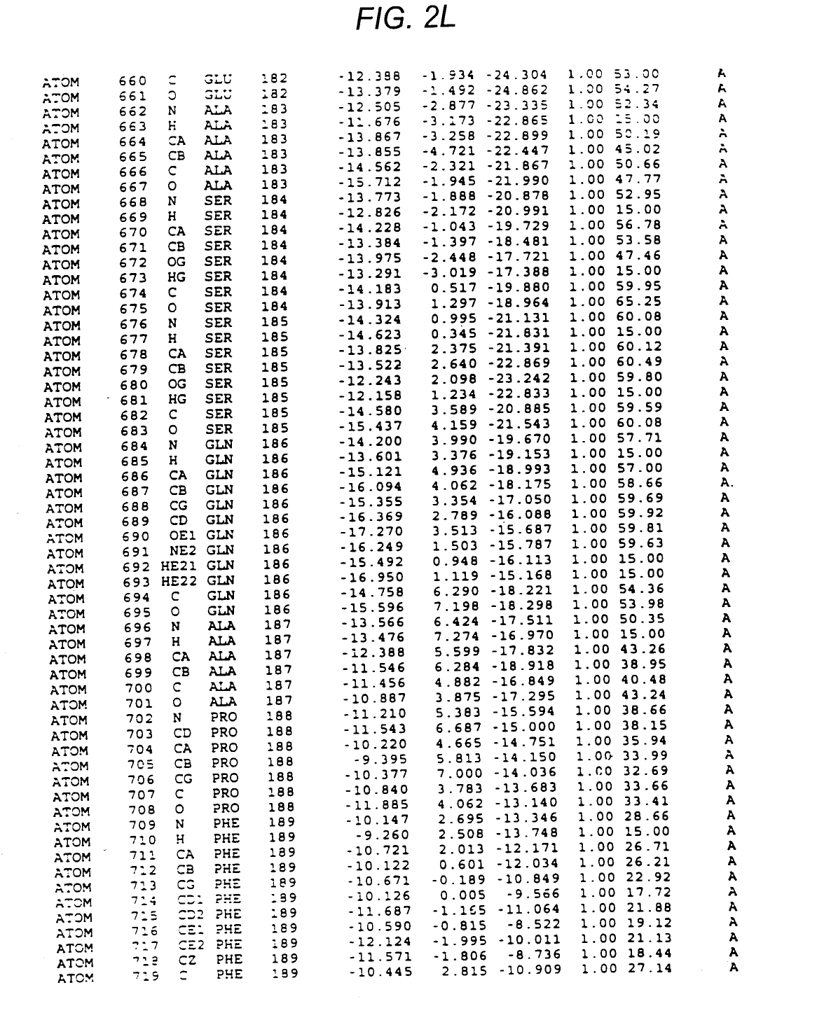

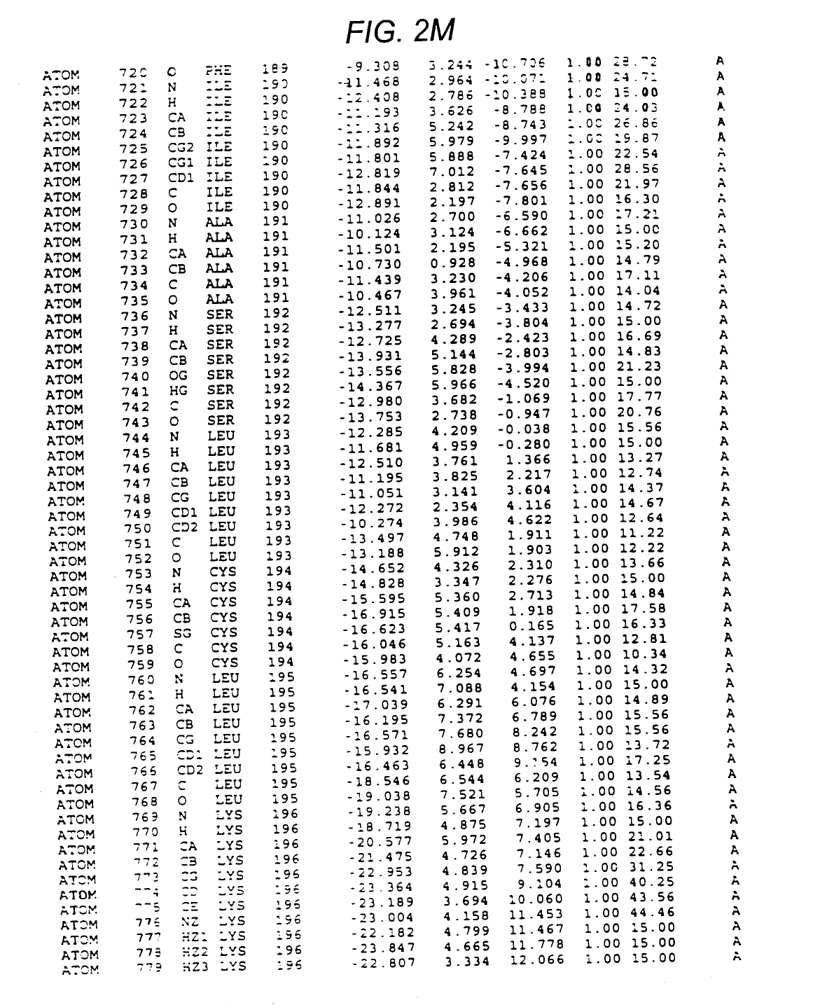

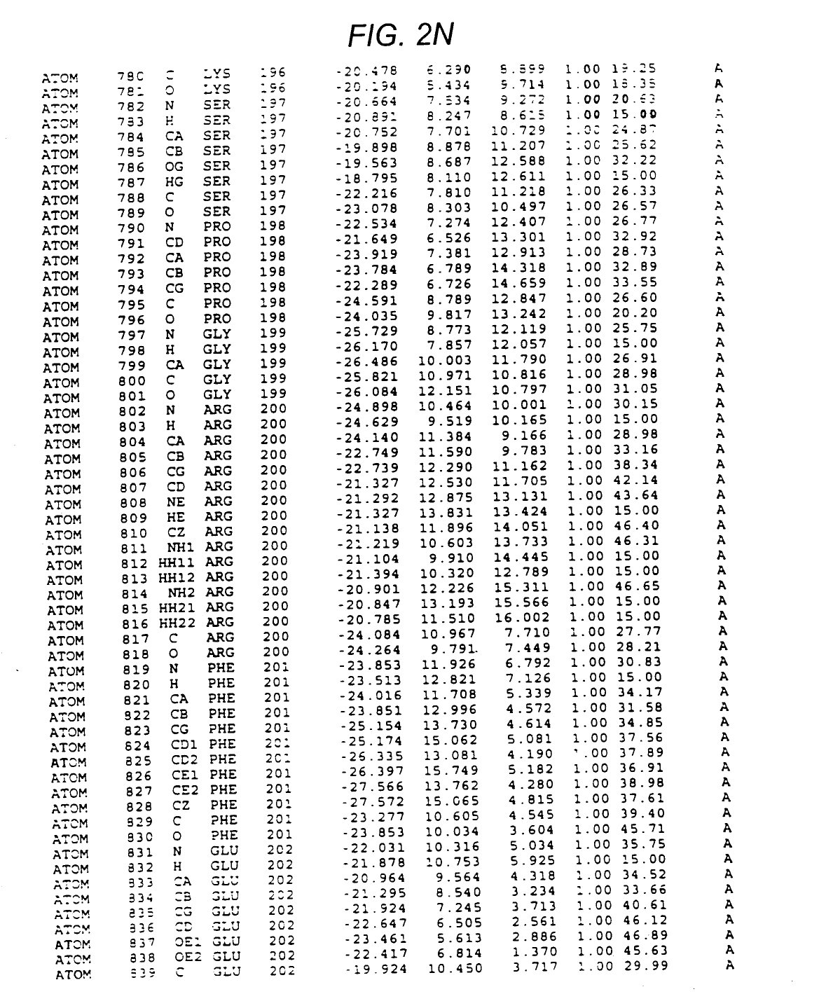

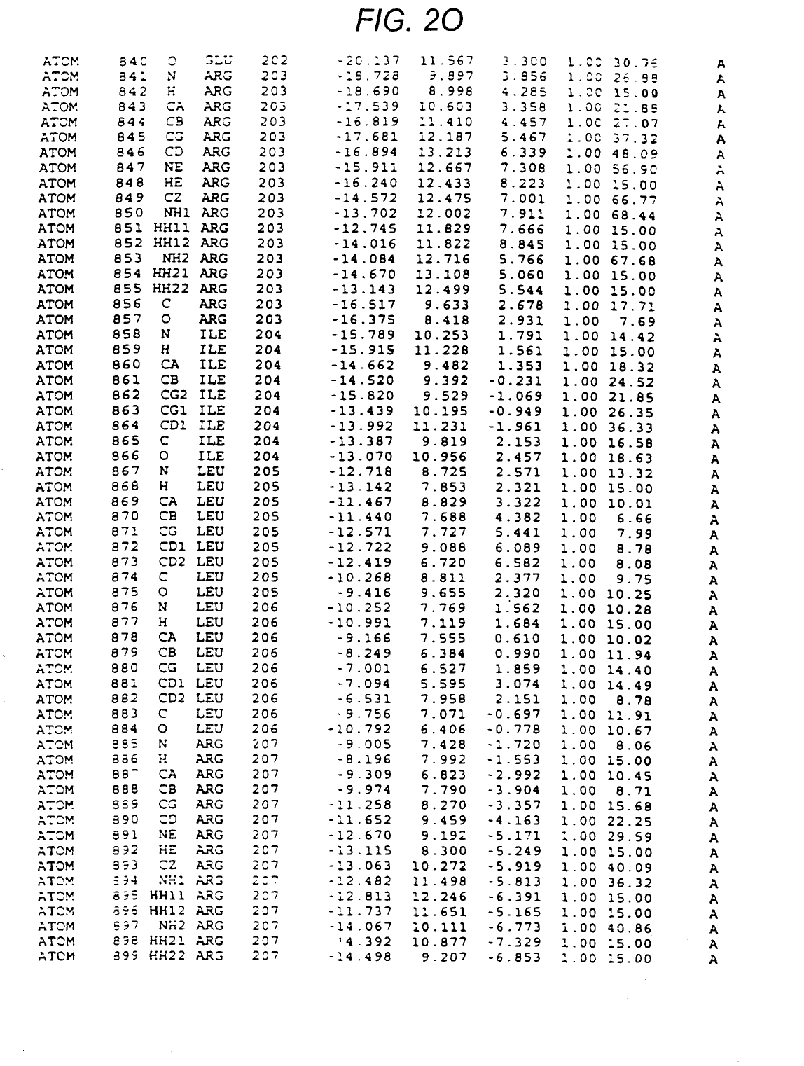

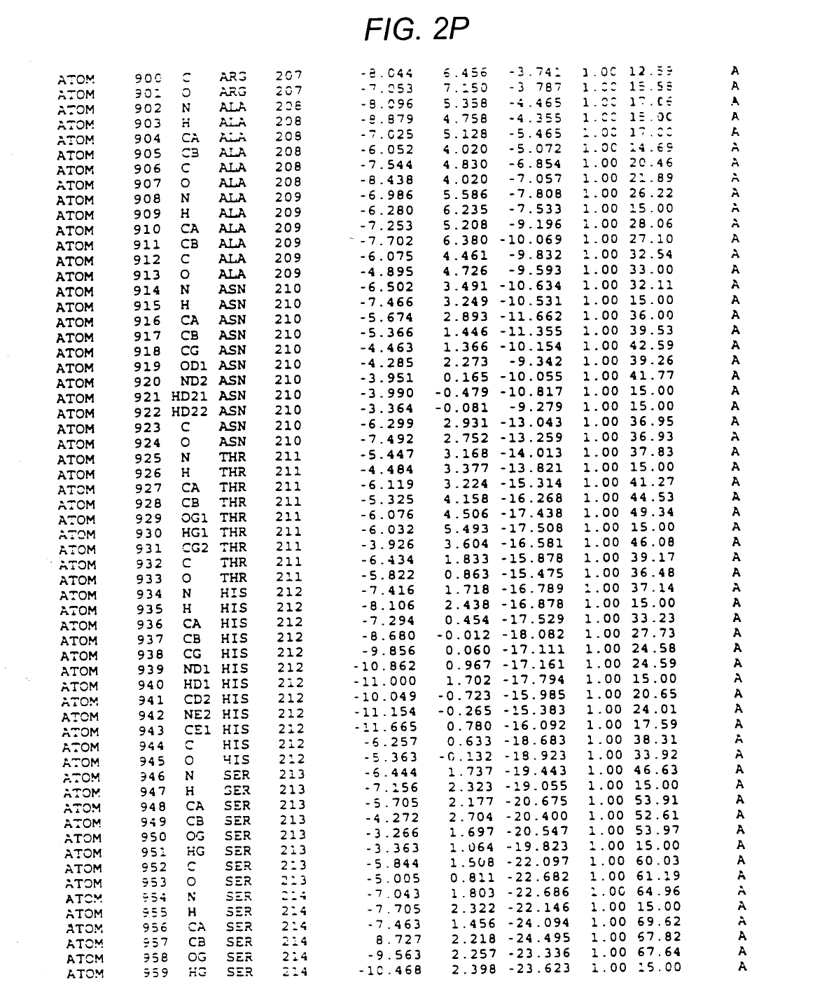

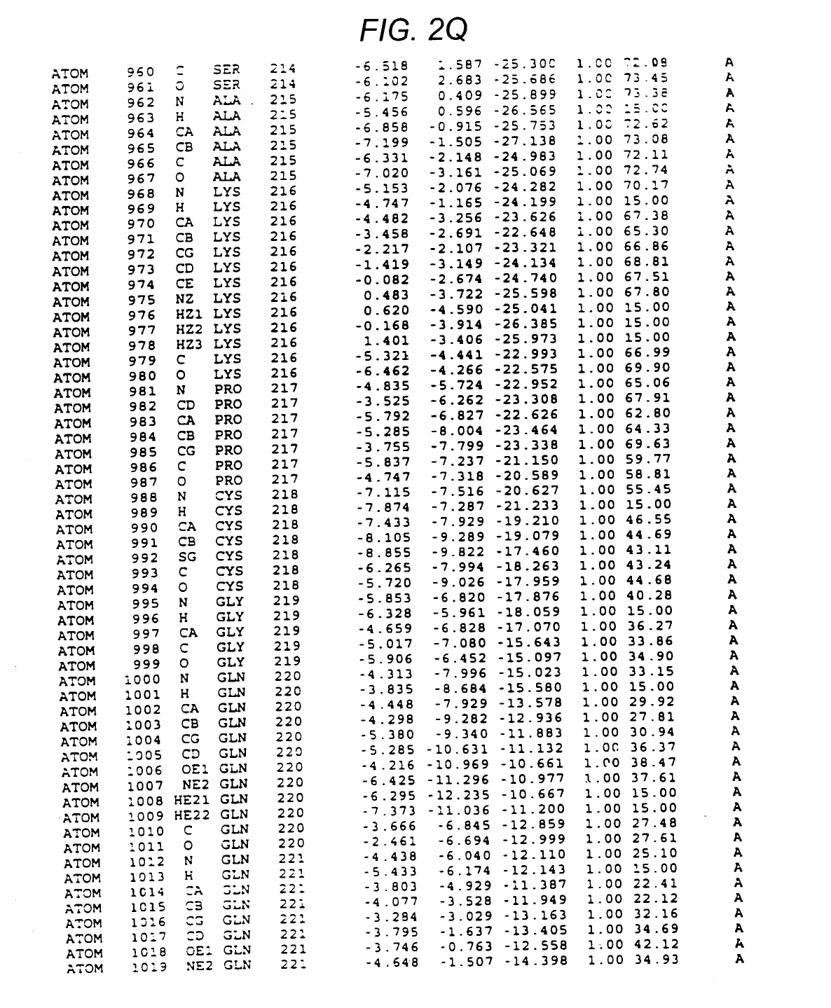

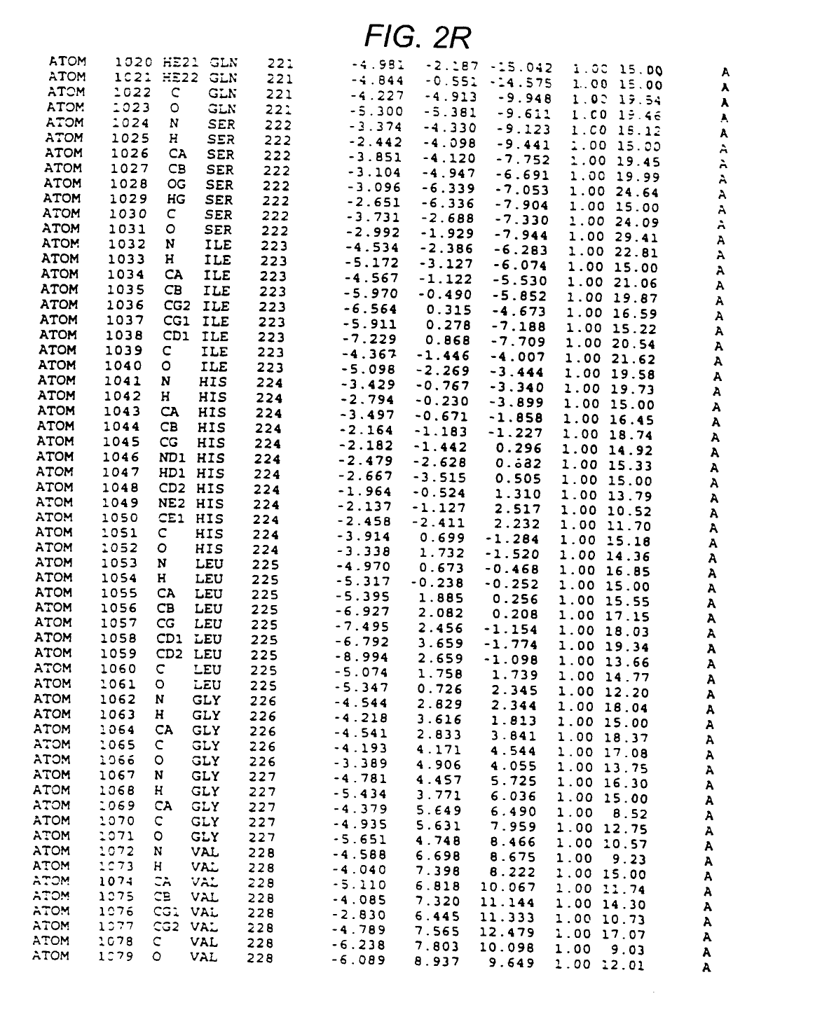

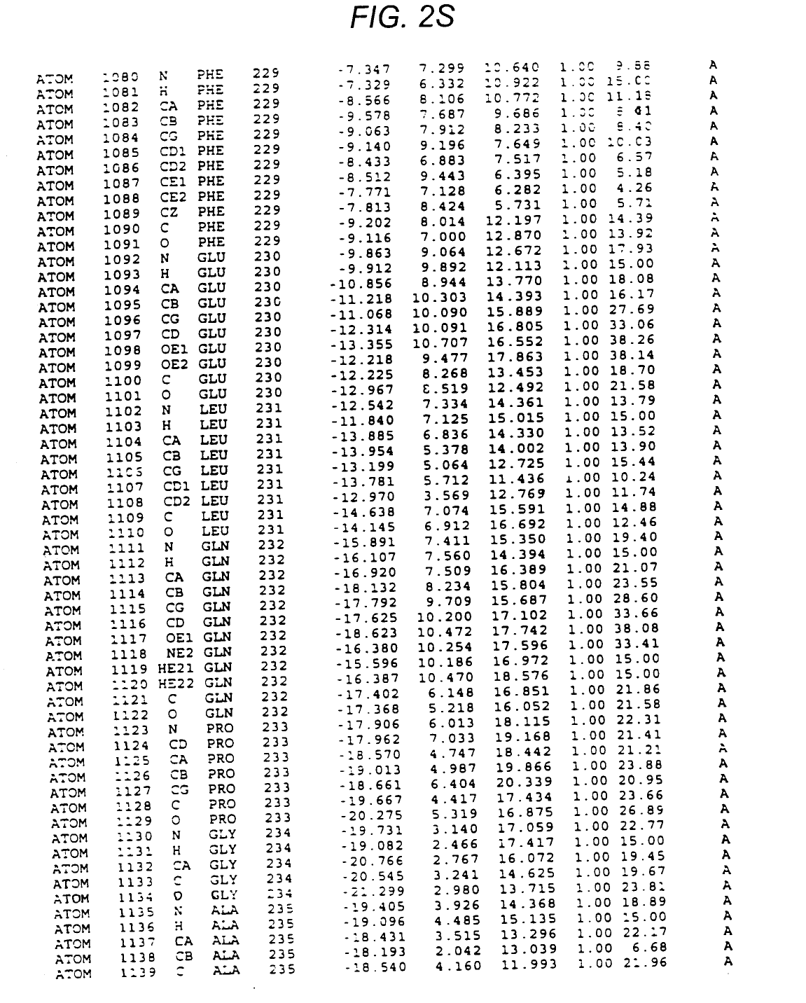

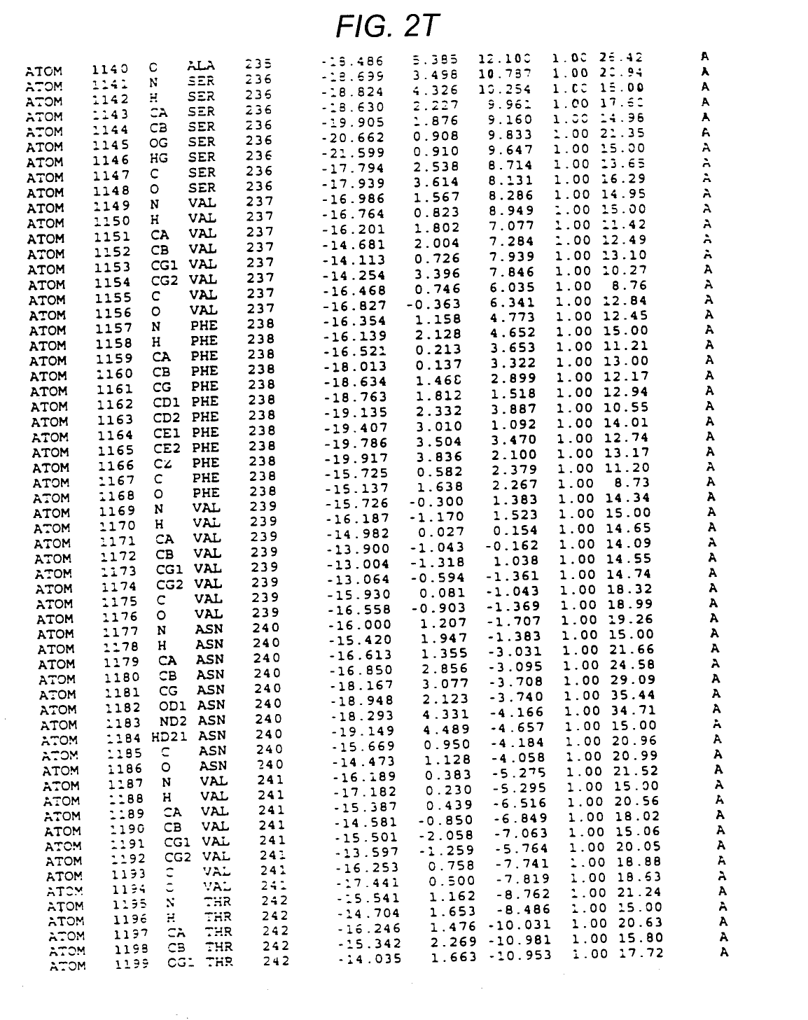

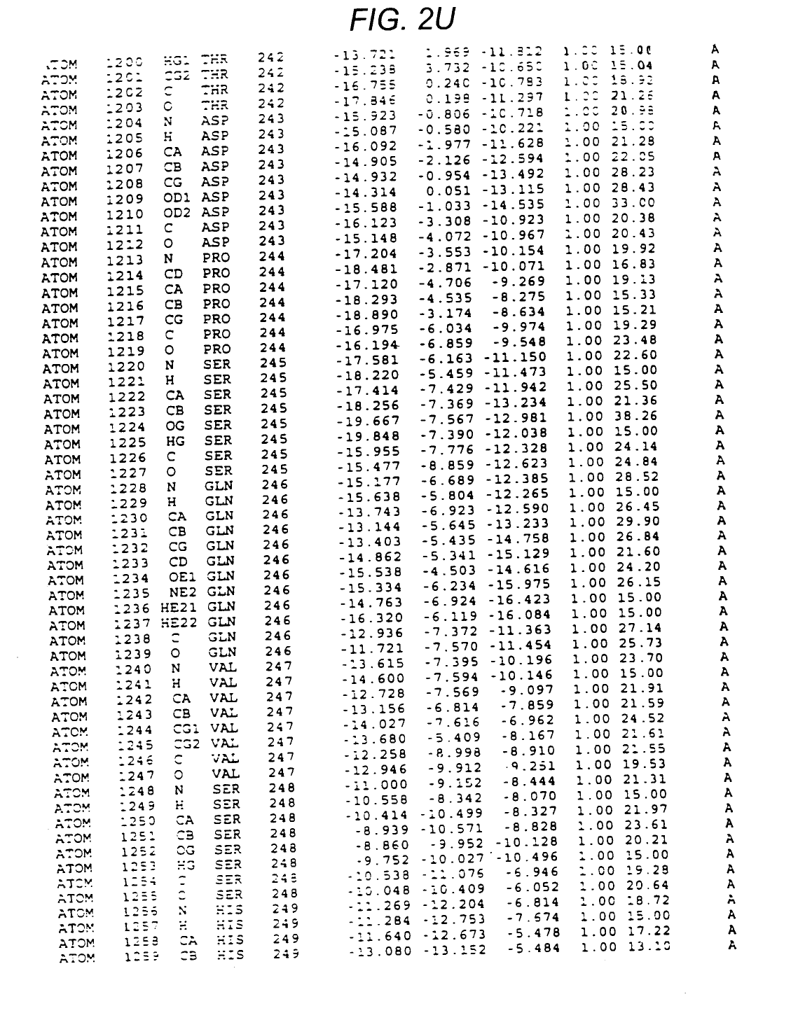

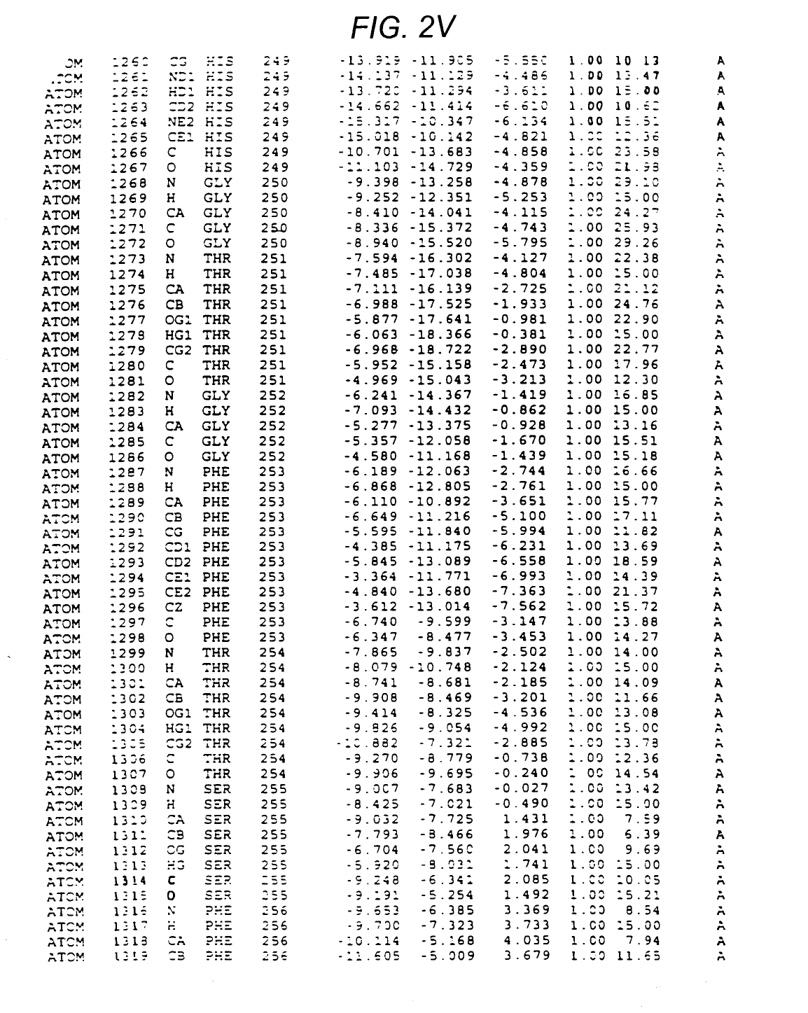

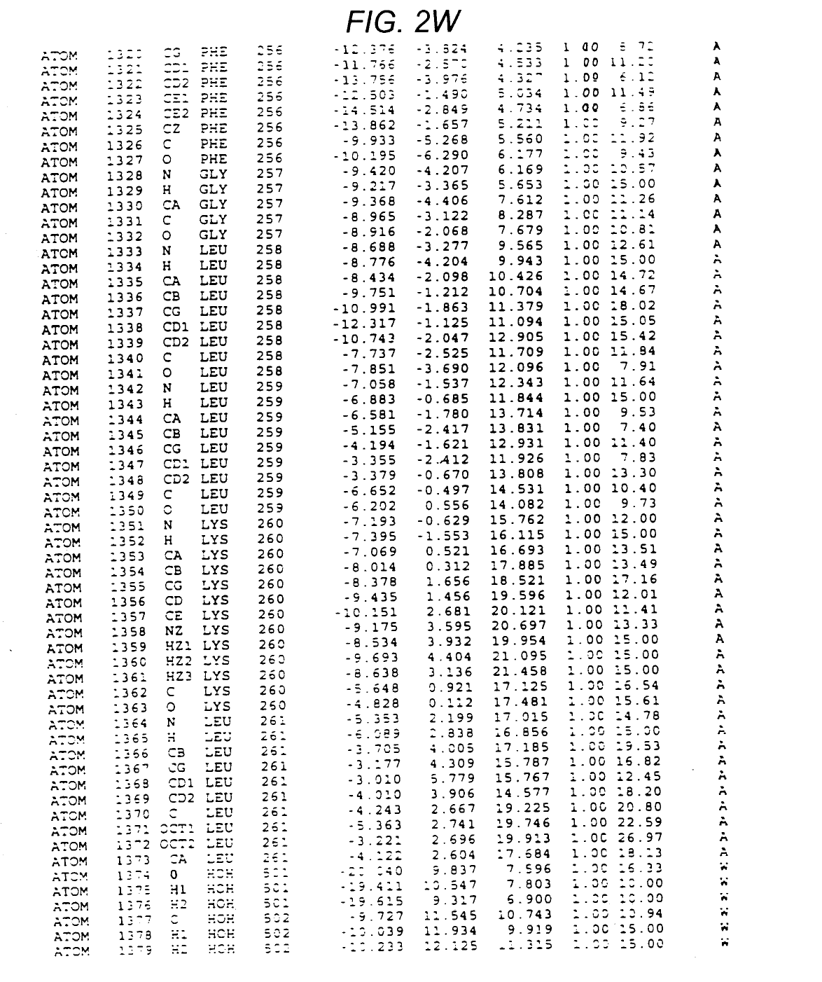

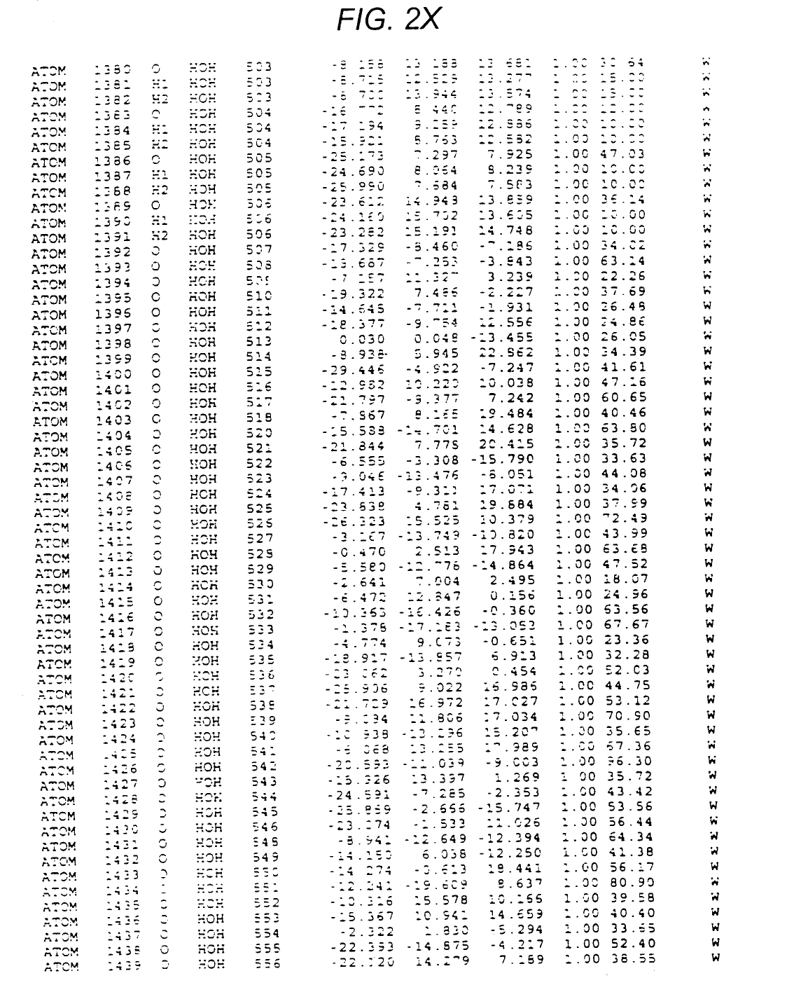

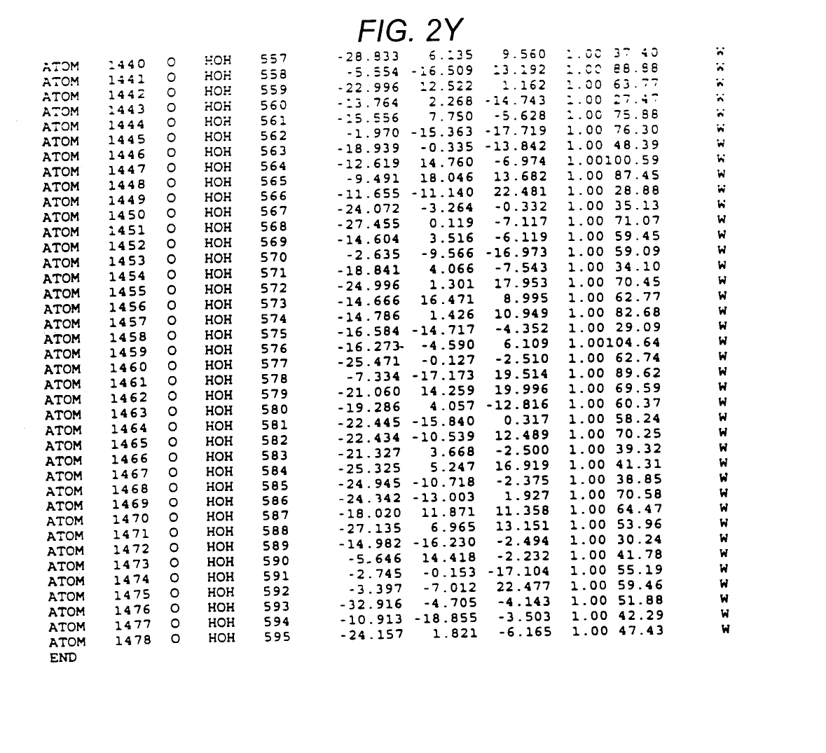

The crystal structure to be used with the screening method has been determined at 2 Å resolution by the method of molecular replacement. In brief, a soluble fragment of the extracellular domain of human CD40 ligand containing amino acid residues Gly 116 to the c-terminal residue Leu 261 was first produced in soluble form, then purified and crystallized. The crystals were used to collect diffraction data. Molecular replacement and refinement were done with the XPLOR program package and QUANTA (Molecular Simulations, Inc.) Software. In particular, a 3-dimensional model of human sCD40L was constructed using the murine CD40L model using QUANTA protein homology modeling software. This model was used as a probe for crystallographic analysis calculations and refined using XPLOR. This method of determining the crystal structure of sCD40L is described in more detail in Karpusas et al., “2 Å crystal structure of an extracellular fragment of human CD40 ligand,” Structure (October 1995) 3(10):1031-1039. The atomic coordinates of sCD40L(116-261) are provided in FIGS. 2A-Y. The screening method for selecting an agent includes computational drug design and iterative structure optimization, as described below.

The agent may be an inhibitor selected using computational drug design. Using this method, the sCD40L crystal structure coordinates are used as an input for a computer program, such as DOCK, which outputs a list of molecular structures that are expected to bind to CD40L. Use of such computer programs is well-known. See, e.g., Kuntz, “Structure-Based Strategies for drug design and discovery,” Science, vol. 257, p. 1078 (1992). The list of molecular structures can then be screened by biochemical assays for CD40L binding. Competition-type biochemical assays, which are well known, can be used. See, e.g., Bajorath et al., “Identification of residues of CD40 and its ligand which are critical for the receptor-ligand interaction,” Biochemistry, 34, p. 1833 (1995). The structures that are found to bind to CD40L can thus be used as agents for the present invention. The agent may also be a modified or designed molecule, determined by interactive cycles of structure optimization. Using this approach, a small molecule inhibitor of CD40L found using the above computational approach or other approach can be co-crystallized with sCD40L and the crystal structure of the complex solved by molecular replacement. The information revealed through molecular replacement can be used to optimize the structure of the inhibitors by clarifying how the molecules interact with CD40L. The molecule may be modified to improve its physiochemical properties, including specificity and affinity for CD40L.

In an embodiment of this invention the agent is a small molecule. As used herein a small molecule is a compound having a molecular weight between 20 Da and 1×10⁶ Da, preferably from 50 Da to 2 kDa.

This invention also provides a method of inhibiting activation by CD40 ligand of smooth muscle cells bearing CD40 on the surface of the cells, in a subject, comprising administering to the subject an agent capable of inhibiting interaction between CD40 ligand and CD40 on the cells, the agent being present in an amount effective to inhibit activation of the cells in the subject.

In specific embodiments the CD40-bearing smooth muscle cells are smooth muscle cells of the bladder, vascular smooth muscle cells, bronchial smooth muscle cells, aortic smooth muscle cells, coronary smooth muscle cells, pulmonary smooth muscle cells, or gastrointestinal smooth muscle cells. In more specific embodiments the gastrointestinal smooth muscle cells are esophageal, stomachic, or intestinal smooth muscle cells, including smooth muscle cells of the small intestine or large intestine (bowel).

In an embodiment of this invention the agent inhibits binding of CD40 ligand to CD40 on the cells.

In an embodiment of this invention the agent is a protein. In another embodiment of this invention the agent is a nonprotein.

In a specific embodiment the protein comprises an antibody or portion thereof capable of inhibiting interaction between CD40 ligand and CD40 on the cells. The antibody is a monoclonal or polyclonal antibody. In a more specific embodiment the monoclonal antibody specifically binds to the epitope to which monoclonal antibody 5c8 (ATCC Accession No. HB 10916) specifically binds. An example of such a monoclonal antibody is monoclonal antibody 5c8 (ATCC Accession No. HB 10916). In other embodiments the monoclonal antibody is a chimeric antibody or a humanized antibody.

In a specific embodiment the portion of the antibody comprises a complementarity determining region or variable region of a light or heavy chain. In another specific embodiment the portion of the antibody comprises a complementarity determining region or a variable region. In another specific embodiment the portion of the antibody comprises a Fab or a single chain antibody.

In another embodiment the protein comprises soluble extracellular region of CD40 ligand or portion thereof capable of inhibiting interaction between CD40 ligand and CD40 on the cells; or soluble extracellular region of CD40 or portion thereof capable of inhibiting interaction between CD40 ligand and CD40 on the cells. In a specific embodiment the soluble extracellular region of CD40 ligand or CD40 is a monomer. In another embodiment the soluble extracellular region of CD40 is an oligomer.

In another embodiment of this invention the protein comprising soluble extracellular region of CD40 or portion thereof further comprises an Fc region fused to the extracellular region of CD40 or portion thereof. In a specific embodiment the Fc region is capable of binding to protein A or protein G. In another specific embodiment the Fc region comprises IgG, IgG₁, IgG₂, IgG₃, IgG₄, IgA, IgA₁, IgA₂, IgM, IgD, or IgE.

When administered, proteins are often cleared rapidly from the circulation and may therefore elicit relatively short-lived pharmacological activity. Consequently, frequent injections of relatively large doses of bioactive proteins may by required to sustain therapeutic efficacy. Proteins modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified proteins (Abuchowski et al., In: “Enzymes as Drugs”, Holcenberg et al., eds. Wiley-Interscience, New York, N.Y., 367-383 (1981; Anderson, W. F. (1992) Human Gene Therapy. Science 256:808-813.; Newmark et al., (1982) J. Appl. Biochem. 4:185-189; and Katre et al., Proc. Natl. Acad. Sci. USA 84:1487-1491 (1987)). Such modifications may also increase the protein's solubility in aqueous solution, eliminate aggregation, enhance the physical and chemical stability of the protein, and greatly reduce the immunogenicity and antigenicity of the protein. As a result, the desired in vivo biological activity may be achieved by the administration of such polymer-protein adducts less frequently or in lower doses than with the unmodified protein.

Attachment of polyethylene glycol (PEG) to proteins is particularly useful because PEG has very low toxicity in mammals (Carpenter et al., 1971). For example, a PEG adduct of adenosine deaminase was approved in the United States for use in humans for the treatment of severe combined immunodeficiency syndrome. A second advantage afforded by the conjugation of PEG is that of effectively reducing the immunogenicity and antigenicity of heterologous proteins. For example, a PEG adduct of a human protein might be useful for the treatment of disease in other mammalian species without the risk of triggering a severe immune response. In one embodiment of this invention, the protein may be delivered in a microencapsulation device so as to reduce or prevent an host immune response against the protein. The protein may also be delivered microencapsulated in a membrane, such as a liposome.

Polymers such as PEG may be conveniently attached to one or more reactive amino acid residues in a protein such as the alpha-amino group of the aminoterminal amino acid, the epsilon amino groups of lysine side chains, the sulfhydryl groups of cysteine side chains, the carboxyl groups of aspartyl and glutamyl side chains, the alpha-carboxyl group of the carboxy-terminal amino acid, tyrosine side chains, or to activated derivatives of glycosyl chains attached to certain asparagine, serine or threonine residues.

Numerous activated forms of PEG suitable for direct reaction with proteins have been described. Useful PEG reagents for reaction with protein amino groups include active esters of carboxylic acid or carbonate derivatives, particularly those in which the leaving groups are N-hydroxysuccinimide, p-nitrophenol, imidazole or 1-hydroxy-2-nitrobenzene-4-sulfonate. PEG derivatives containing maleimido or haloacetyl groups are useful reagents for the modification of protein free sulfhydryl groups. Likewise, PEG reagents containing amino hydrazine or hydrazide groups are useful for reaction with aldehydes generated by periodate oxidation of carbohydrate groups in proteins.

The subject which can be treated by the above-described methods is an animal. Preferably the animal is a mammal. Examples of mammals which may be treated include, but are not limited to, humans, non-human primates, rodents (including rats, mice, hamsters and guinea pigs) cow, horse, sheep, goat, pig, dog and cat.

In an embodiment of this invention, the agent is selected by a screening method.

In a specific embodiment the agent is selected by a screening method, which comprises isolating a sample of cells; culturing the sample under conditions permitting activation of CD40-bearing cells; contacting the sample with cells expressing a protein which is specifically recognized by monoclonal antibody 5c8 produced by the hybridoma having ATCC Accession no. HB 10916, or with a protein which is specifically recognized by monoclonal antibody 5c8 produced by the hybridoma having ATCC Accession no. HB 10916, effective to activate the CD40-bearing cells; contacting the sample with an amount of the agent effective to inhibit activation of the CD40-bearing cells if the agent is capable of inhibiting activation of the CD40-bearing cells; and determining whether the cells expressing the protein which is specifically recognized by monoclonal antibody 5c8 produced by the hybridoma having ATCC Accession no. HB 10916, or with the protein which is specifically recognized by monoclonal antibody 5c8 produced by the hybridoma having ATCC Accession no. HB 10916, activate the CD40-bearing cells in the presence of the agent. The cell sample may be isolated from diverse tissues, including cell lines in culture or cells isolated from an animal, such as dispersed cells from a solid tissue, cells derived from a bone marrow bipsy, or cells isolated from a body fluid such as blood or lymphatic fluid.

In another specific embodiment the molecule (agent) is selected based on a three-dimensional structure of soluble extracellular region of CD40 ligand or portion thereof capable of inhibiting interaction between CD40 ligand and CD40 on the cells. The molecule may be selected from a library of known molecules, modified from a known molecule based on the three-dimensional structure, or designed and synthesized de novo based on the three-dimensional structure. In specific embodiments the agent or molecule is designed by structure optimization of a lead inhibitory agent based on a three-dimensional structure of a complex of the soluble extracellular region of CD40 ligand or portion thereof with the lead inhibitory agent.

Method of Treatment

This invention provides a method of treating, in a subject, a smooth muscle cell-dependent disease, comprising the above-described method of inhibiting activation by CD40 ligand of smooth muscle cells bearing CD40 on the surface of the cells, which comprises administering to the subject an agent capable of inhibiting interaction between CD40 ligand and CD40 on the cells, the agent being present in an amount effective to inhibit activation of the cells in the subject.

In an embodiment of this invention the smooth muscle cell-dependent disease is a vascular disease. In a specific embodiment the vascular disease is atherosclerosis.

In another embodiment the smooth muscle cell-dependent disease is a gastrointestinal disease. In a specific embodiment the gastrointestinal disease is selected from the group consisting of esophageal dysmotility, inflammatory bowel disease, and scleroderma.

In an embodiment the smooth muscle cell-dependent disease is a bladder disease.

The compounds of this invention may be administered in any manner which is medically acceptable. This may include injections, by parenteral routes such as intravenous, intravascular, intraarterial, subcutaneous, intramuscular, intratumor, intraperitoneal, intraventricular, intraepidural, or others as well as oral, nasal, ophthalmic, rectal, topical, or inhaled. Sustained release administration is also specifically included in the invention, by such means as depot injections of erodible implants directly applied during surgery.

The compounds are administered at any dose per body weight and any dosage frequency which is medically acceptable. Acceptable dosage includes a range of between about 0.01 and 200 mg/kg subject body weight. A preferred dosage range is between about 0.1 and 50 mg/kg. Particularly preferred is a dose of between about 1 and 30 mg/kg. The dosage is repeated at intervals ranging from each day to every other month. One preferred dosing regimen is to administer a compound of the invention daily for the first three days of treatment, after which the compound is administered every 3 weeks, with each administration being intravenously at 5 or 10 mg/kg body weight. Another preferred regime is to administer a compound of the invention daily intravenously at 5 mg/kg body weight for the first three days of treatment, after which the compound is administered subcutaneously or intramuscularly every week at 10 mg per subject. Another preferred regime is to administer a single dose of the compound of the invention parenterally at 20 mg/kg body weight, followed by administration of the compound subcutaneously or intramuscularly every week at 10 mg per subject.

The compounds of the invention may be administered as a single dosage for certain indications such as preventing immune response to an antigen to which a subject is exposed for a brief time, such as an exogenous antigen administered on a single day of treatment. Examples of such an antigen would include coadministration of a compound of the invention along with a gene therapy vector, or a therapeutic agent such as an antigenic pharmaceutical or a blood product. In indications where antigen is chronically present, such as in controlling immune reaction to transplanted tissue or to chronically administered antigenic pharmaceuticals, the compounds of the invention are administered at intervals for as long a time as medically indicated, ranging from days or weeks to the life of the subject.

Inflammatory responses are characterized by redness, swelling, heat and pain, as consequences of capillary dilation with edema and migration of phagocytic leukocytes. Inflammation is further defined by Gallin (Chapter 26, Fundamental Immunology, 2d Ed., Raven Press, New York, 1989, pp. 721-733), which is herein incorporated by reference.

This invention will be better understood from the Experimental Details which follow. However, one skilled in the art will readily appreciate that the specific methods and results discussed are merely illustrative of the invention as described more fully in the claims which follow thereafter.

Experimental Details

Examples 1 and 2 below demonstrate that inflammatory cytokines induce smooth muscle cells to express CD40. Moreover, they demonstrate that CD40L mediated signals regulate smooth muscle cell functions.

### EXAMPLE 1

FACS analysis was utilized to investigate if smooth muscle cells express CD40. In 6 well plates human aortic smooth muscle cells were cultured in M199 media supplemented with 25% FCS, 5% human serum, heparin 90 μg/ml, endothelial cell growth factor 15 μg/ml, and 1% penicillin-streptomycin. The media was changed every 2-3 days and when the cells were near confluent they were cultured in the presence or absence of IFN-γ (1000 U/cc), IL-1α (1 ng/cc) or TNF-α (200 U/cc) for 72 hours. The cells were collected by trypsin-EDTA treatment and CD40 expression determined by FACS analysis utilizing anti-CD40 mAb G28.5. The cells were also stained with an isotype negative control mAb and anti-CD54 (ICAM-1) mAb was utilized as a positive control.

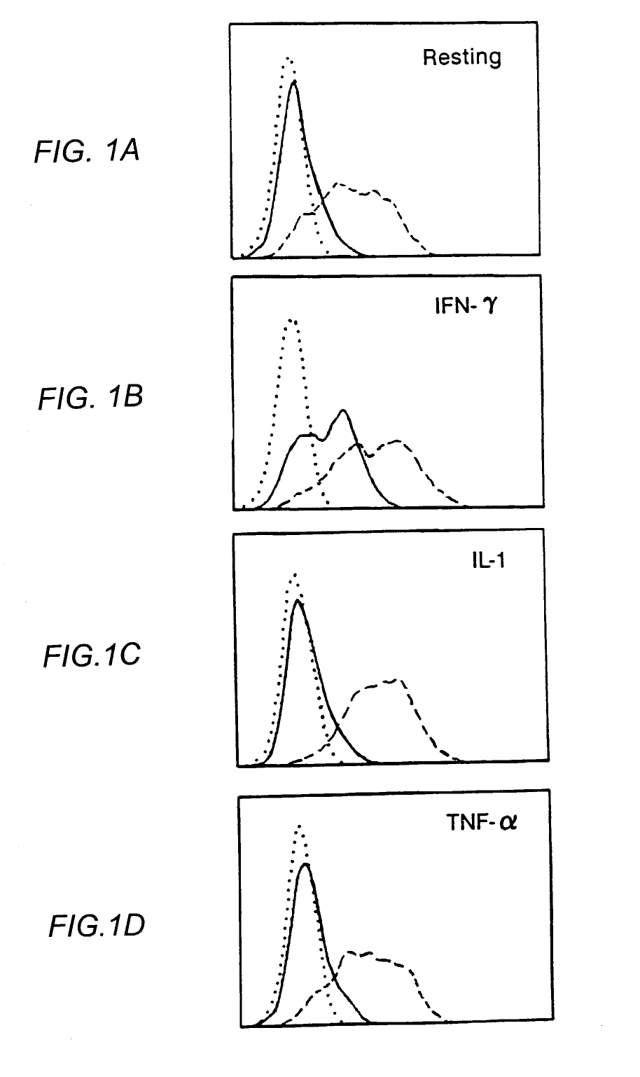

Smooth muscle cells do not constitutively express CD40 as demonstrated in FIG. 1A. However, IFN-γ in contrast to IL-1α or TNF-α, upregulates smooth muscle cell CD40 expression (FIGS. 1A, 1B, and 1C). These studies demonstrate that IFN-γ upregulates CD40 expression on human aortic smooth muscle cells.

### EXAMPLE 2

CD40 expression on smooth muscle cell was examined in situ. Cells found in the media of normal vessels which morphologically resemble smooth muscle cells do not react with anti-CD40 mAb. However, cells which morphologically resemble smooth muscle cells found within inflammatory lesions in accelerated atherosclerosis associated with transplantation express CD40 in situ. These studies suggest that inflammatory cytokines induce smooth muscle cells to express CD40. Moreover, these studies demonstrate that CD40L-mediated signals regulate smooth muscle cell functions.

### EXAMPLE 3

CD40L⁺ CD4⁺ T Cells and CD40⁺ Target Cells are Present in Atherosclerosis and Transplant Coronary Artery Disease.

Activated endothelial cells (EC), macrophages (Mac) and CD4⁺ T cells are present early in the lesions of coronary atherosclerosis (CA) and cardiac transplant atherosclerosis (TA). Because CD40L is an activation-induced CD4⁺ T cell surface molecule that delivers contact-dependent activating signals to CD40⁺ target cells including EC (upregulated ICAM, VCAM and E-selectin expression) and Mac (induces NO, TNF-α and IL-1 production), we investigated in situ CD40L and CD40 expression in CA (n=5) and TA (n=5). CD40L and CD40 expression was determined utilizing anti-CD40L mAb 5C8, anti-CD40 mAb G28.5 or appropriate control mAbs. Frozen sections of normal coronary arteries (n=3) do not contain T cells and CD40 expression is restricted to EC. In contrast, lesions associated with CA and TA contain CD40L⁺ CD4⁺ T cells as determined by immunolabelling of serial sections. Additionally, CD40 expression in frozen sections from patients with CA and TA is markedly upregulated on EC, infiltrating mononuclear cells, foam cells and intimal smooth muscle cells (SMC). Two color immunohitochemical analysis of paraffin fixed tissue utilizing SMC (smooth muscle actin ) or Mac (HAM-56) specific markers confirm the expression of CD40 on these cells. Interestingly, intimal SMC distant from inflammatory cells and medial SMC are CD40⁻, suggesting that local inflammatory mediators upregulate CD40 expression on SMC in vivo. CD40 upregulation and CD40L⁺ CD4⁺ T cells are found in all stages of TA and are most marked in early lesions of CA, including fatty streaks. Together, these studies suggest that CD40L⁺ T cells may interact with CD40⁺ target cells in CA and TA and contribute to the pathogenesis of these diseases by promoting production of proinflammatory molecules.

### EXAMPLE 4

CD40 is Expressed on Smooth Muscle Cells and Macrophages in Lesions of Transplant Atherosclerosis.

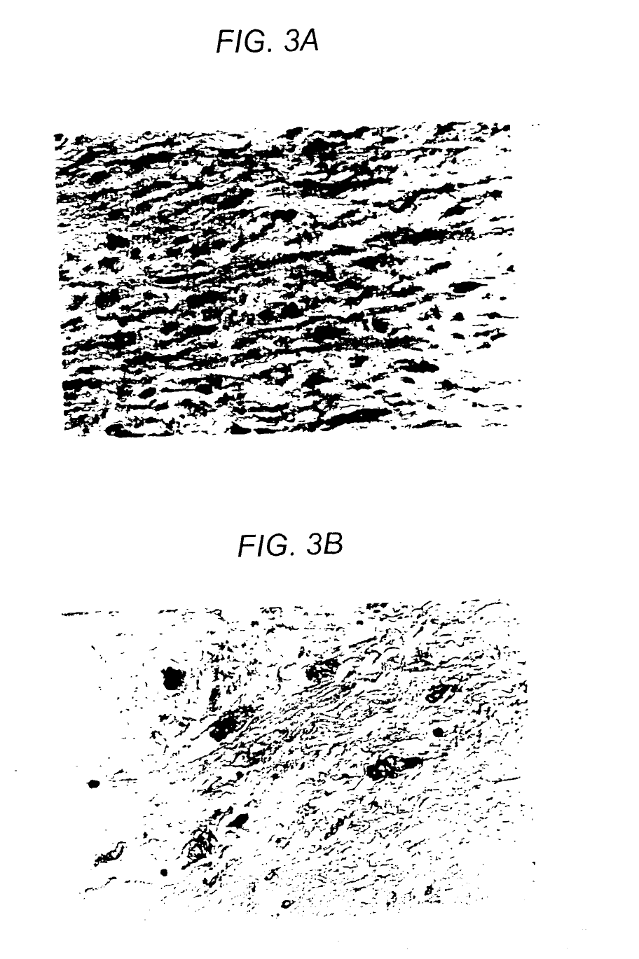

In situ CD40 expression in native atherosclerosis or transplant associated atherosclerosis was studied by two color immunohistochemical analysis. Double labeling immunohistochemistry studies were performed on coronary arteries that had been fixed in 10% buffered formalin and paraffin embedded. Sections were deparaffinized in xylene, hydrated and endogenous peroxidase quenched with ⅕% H₂O₂ in 80% alcohol. Sections were then digested with 0.01% pepsin in HCl (pH 1.5) for 15 minutes at 37° C. Sections were then rinsed in PBS and incubated with 10% horse serum for 20 minutes to block non-specific staining. Then anti-CD40 staining was detected with the Vector ABC Elite Kit (Vector) sequentially utilizing a biotinylated secondary antibody, avidin-peroxidase complex and 3,3′ diaminobenzidine as developer. The presence of CD40 was noted as brown staining. Thereafter, sections were rinsed in PBS and blocked again with 10% horse serum. Sections were then incubated for 1 hour with mAbs specific for smooth muscle cells (smooth muscle actin) or macrophages (HAM 56). The primary antibodies were then conjugated to alkaline phosphatase using an avidin-biotin system (Vector). Vector Red (Vector) was used to detect alkaline phosphatase activity and staining yielded a red reaction. Hence, double labeled cells stained brown (CD40) and red (smooth muscle cells or macrophages). To control for interference between the two immunohistochemical procedures used for dual labeling analysis, serial sections of each specimen were also stained either for CD40, smooth muscle actin or HAM 56. See FIGS. 3A and 3B. Control sections showed the same distribution of immunoreactivity for each of the primary mAbs as the double stained sections.

### EXAMPLE 5

The Distribution Of CD40L And CD40 in Native Coronary Atherosclerosis and Transplant Associated Coronary Artery Disease: Correlation Of CD40 Expression with the Presence of Intercellular Adhesion Molecules, Activated NF-kB and Presence of T Lymphocytes.

T cells play roles in the pathogenesis of native coronary atherosclerosis (CA) and transplant associated coronary artery disease (TCAD), however the mechanisms by which T cells interact with other cells in these lesions are not fully known. CD40L is an activation-induced CD4+ T cell surface molecule that interacts with CD40+ target cells, including macrophages and endothelial cells, and induces the production of proinflammatory molecules, including ICAM-1 and VCAM-1. Moreover, ligation of CD40 is known to activate the transcription factor NF-kB. To investigate whether CD40L-CD40 interactions may play roles in the pathogenesis of CA or TCAD immunohistochemical studies were performed of CD40L and CD40 expression on frozen sections of coronary arteries obtained from cardiac allograft recipients with CA (n=10) or TCAD (n=9). Utilizing two different anti-CD40L mAb it was found that CD40L expression was restricted to infiltrating lymphocytes in CA and TCAD. CD40 expression was markedly upregulated on intimal endothelial cells, foam cells, macrophages and smooth muscle cells in both diseases. Dual immunolabelling demonstrated many CD40+ cells co-expressed ICAM-1, VCAM-1 or the activated form of NF-kB. The extent of CD40, ICAM-1 and VCAM-1 expression showed statistical significant correlation with the severity of disease and the amount of intimal lymphocytes. Together these studies demonstrate the presence of activated CD40L+ and CD40⁺ cells in both CA and TCAD lesions and suggest that CD40L mediated interactions with CD40+ macrophages, foam cells, smooth muscle cells and/or endothelial cells may contribute to the pathogenesis of these diseases.

Several lines of evidence indicate that cell-mediated immune mechanisms contribute to the inflammatory lesions (1-4) characteristic of native coronary atherosclerosis (CA) (5-10) and transplant-associated coronary artery disease (TCAD) (11-13). For example, infiltrating intimal T cells expressing activation markers such as CD25 and MHC Class II molecules are present early in the development of the vascular lesions of both diseases (5, 14). Activated macrophages are commonly found in lesions of both diseases, as are cytokines associated with T cell dependent immune responses, including IFN-γ, IL-1 and TNF-α (5-17). As further evidence that T cells may play pathogenic roles in CA, CD4⁺ T cell clones have been isolated from human fibroatheromatous CA plaques that proliferate and secrete IFN-γ when presented with oxidized LDL (18), a major constituent of the lesions of both native CA and TCAD (1, 19, 20). Furthermore, hyperlipidemia induced atherosclerotic lesions are reduced in mice treated with anti-CD4 mAbs (21). Similarly, vascular lesions of TCAD are significantly ameliorated when allografts were placed in strains of mice genetically deficient in T cells (13) or treated with anti-CD413 or anti-IFN-γ mAbs (22). Together these data strongly suggest that T cells and T cell-derived effector molecules are involved in the pathogenesis of these diseases (9, 23, 24). CD40L is a 30-33 kDa MW surface molecule expressed on activated CD4⁺ T cells which delivers contact-dependent signals to CD40⁺ target cells, such as B cells (25-29). CD40L mediated signals are critically important in the development of T cell dependent humoral immune responses in vitro and in vivo (30). CD40L-CD40 interactions are now known to also play roles in cell mediated immune responses in vitro and in vivo (31, 32). Interestingly, macrophages and endothelial cells, cell types known to participate in the pathogenesis of CA and TCAD, also express CD40 (33-37). Moreover, ligation of CD40 on macrophages and endothelial cells in vitro induces the production of molecules that enhance immune responses and/or have pro-inflammatory effects. For example, CD40L-CD40 interactions upregulate expression of MHC Class II and the costimulatory molecule CD86 on macrophages in vitro (38). Furthermore, ligation of CD40 on macrophages induces the production of cytokines (TNF-α, IL-1β, IL-12), chemokines (IL-8, MIP-1α), nitric oxide (NO) via induction of NO synthese 2, the procbagulant protein tissue factor and matrix metalloproteinases (33, 34, 39-42). CD40L-CD40 interactions upregulate intercellular adhesion molecules CD54 (ICAM-1), CD106 (VCAM-1) and CD62E (E-selectin) on endothelial cells (35-37). Many of the effects of CD40 ligation are dependent on activation of the transcription factor NF-κB (43-45).

Together these findings suggest the notion that ligation of CD40 on a variety of target cells may augment CD4⁺ T cell mediated inflammatory reaction in vivo. In support of this hypothesis, CD40 expression is upregulated in the kidneys of patients with lupus glomerulonephritis, IgA nephropathy and ANCA⁺ glomerulonephritis and in the skin of patients with psoriasis (35, 46). Moreover, CD40L⁺ T cells infiltrate the kidneys of patients with inflammatory renal diseases (46). Because interactions of T cells with macrophages, endothelial cells and possibly other cells play roles in the pathogenesis of CA and TCAD, in the current study the expression of CD40L and CD40 in these two diseases is investigated using immunohistochemistry. CD40L is expressed on T cells and CD40 expression is upregulated on endothelial cells, smooth muscle cells, macrophages and “foam” cells in the intimal lesions of both diseases. Moreover, using double immunostaining it is found that many CD40⁺ cells in these lesions co-express CD54, CD106 and the activated form of NF-κB.

Methods: Human Coronary Arteries

Segments from the main left coronary artery or the proximal portion of the left anterior descending artery were obtained from the explanted hearts of 23 cardiac allograft recipients. Nine patients underwent retransplantation because they had developed severe transplant-associated coronary artery disease (TCAD). In these patients survival of the first allograft had ranged between 38 and 103 months. Ten patients received cardiac allografts because they had developed severe coronary artery disease and ischemic cardiomyopathy. Control coronary arteries without atherosclerotic changes were obtained from explanted hearts of 4 patients; 3 had idiopathic cardiomyopathy, one a cardiac sarcoma. Portions of each vessel were snap frozen in isopentane at −80° C. and serial sections were cut on a cryostat (Reichert Histostat) at 4 mm thickness. Sections were mounted on sialin coated slides, air dried, fixed in cold acetone for 1 minute, in a 1:1 mixture of cold acetone/chloroform for an additional 7 minutes and stored at −80° C. One section from each coronary artery was fixed in 10% formalin and stained with hematoxylin and eosin for histologic evaluation.

Primary Antibodies

Anti-CD40 hybridoma G28.5 (IgG1) was purchased from American Type Culture Collection (Rockville, Md.). Anti-CD40L mAb 5C8 (IgG2a) was generated as previously described (28). Both G28.5 and 5C8 mAbs were purified from ascites utilizing a protein G column (Pharmacia, Piscataway, N.J.). An additional anti-CD40L mAb (IgG1) was purchased from Calbiochem (San Diego, Calif.). An IgM anti-CD40 mAb was obtained from Caltag (Burlingame, Calif.) and was used for dual immunostaining studies. Monoclonal Abs to CD3, CD4, CD8, CD34, CD68 (Novocastra, Burlingham, Calif., all IgG1) and smooth muscle actin (SMA) (DAKO, Carpinteria, Calif., IgG2a), were used to distinguish among the various cell types of intimal plaques, including T cells (CD3, CD4 or CD8), endothelial cells (CD34), macrophages (CD68) and smooth muscle cells (SMA). Anti-ICAM-1 (IgG1) and anti-VCAM-1 (IgG1) mAbs were purchased from CHEMICON™ (Temecula, Calif.). The distribution of activated NF-κB was demonstrated with p65mAb (IgG3) (BOEHRINGER MANNHEIM™) which binds to an epitope on the p65 subunit of NF-κB blocked by IκB and therefore only accessible when NF-κB is activated by dissociation of IκB(47). Isotype control mAb (Mopec 21, 22) were obtained from SIGMA™ (St. Louis, Mo.)

Immunohistochemistry

Frozen sections were washed in phosphate buffered saline (PBS) and endogenous peroxidase was quenched in 0.5% hydrogen peroxide. Sections were “blocked” with 10% goat serum and aggregated human Ig (80 mg/ml) in PBS and then were incubated for one hour with the indicated primary mAb or the respective control mAb. Frozen sections of tonsils with follicular hyperplasia were used as positive controls to determine the optimal dilution of each mAb. Primary mAb bound to target antigen was linked to biotin labelled isotype specific goat anti-mouse IgGl, IgG2a, IgG3 or IgM (Fisher Scientific, Pittsburgh, Pa.), which was then conjugated to avidin-biotin-peroxidase complexes (VECTOR ELITE KIT™, VECTOR™, Burlingham, Calif.). Peroxidase activity was detected by the chromogen (red) 3-amino-9-ethylcarbazole (AEC, VECTOR™, Burlingham, Calif.) and the sections were counterstained with Mayer's hematoxylin (SIGMA™, St. Louis, Mo.).

Double labelling immunohistochemistry was used to identify the cell types expressing CD40 and to determine the distribution of CD40 in relation to ICAM-1, VCAM-1 or activated NF-κB in atherosclerotic lesions. All sections were first immunolabelled with the IgM anti-CD40 mAb. The secondary Ab was a biotinylated goat anti-mouse IgM which was then conjugated to the avidin-biotin-peroxidase complex. The chromogen used to detect the presence of anti-CD40 IgM mAb was 3,3′ diaminobenzidine (brown). The sections were then rinsed thoroughly and incubated with a second primary mAb targeting either a cell specific marker for smooth muscle cells (SMA) or macrophages (CD68), leukocyte adhesion molecules (ICAM-1, VCAM-1) or the activated form of NF-κB. All of these second primary mAbs were either IgG1, IgG2a or IgG3 isotypes. The appropriate isotype specific biotinylated secondary antibody was applied and conjugated to an avidin-biotin-alkaline phosphatase complex (VECTOR™, Burlingham, Calif.). Alkaline phosphatase activity was demonstrated by the chromogen Vector Red (VECTOR™, Burlingham, Calif.). Interference between the sequentially applied staining procedures was avoided by using different immunoenzymatic techniques (peroxidase vs. alkaline phosphatase) and isotype specific secondary Abs for each target antigen. Furthermore, double labelled control sections were prepared in which one of the two primary mAbs was substituted with an isotype matched control mAb.

Semi-Quantitative Analysis of Lesions

The extent of the atherosclerotic lesions in each section was quantitated by the degree of narrowing of the vascular lumen on a scale from 0 to 4 in which 0 indicated no narrowing, 1 less than 25%, 2 less than 50%, 3 less than 90%, and 4 over 90% luminal narrowing. Each coronary artery lesion was also scored for its content of intimal macrophages, smooth muscle cells, foam cells, endothelial cells (neovascularization) 48 and T cells with 0 indicating absence of the respective cell type, 1 rare isolated cells, 2 small collections of cells, 3 focal dense aggregates present, and 4 dense aggregates present throughout the entire plaque. Similarly, the presence of CD40, ICAM-1, and VCAM-1 was scored on a scale from 0 to 4 in which 0 indicates absence of the respective molecule, 1 its presence on rare cells, 2 its presence on less than 50%, 3 on less than 90%, and 4 on more than 90% of all cells (49). Because the expression of CD40L in positive specimens was limited to isolated cells its presence was not amenable to quantitative evaluation.

Statistical Analysis

Differences in histological scores among groups of specimens were analyzed using the non parametric Kruskal Wallis procedure. The association between variables was assessed using Spearman's correlation.

Results: Normal Coronary Arteries

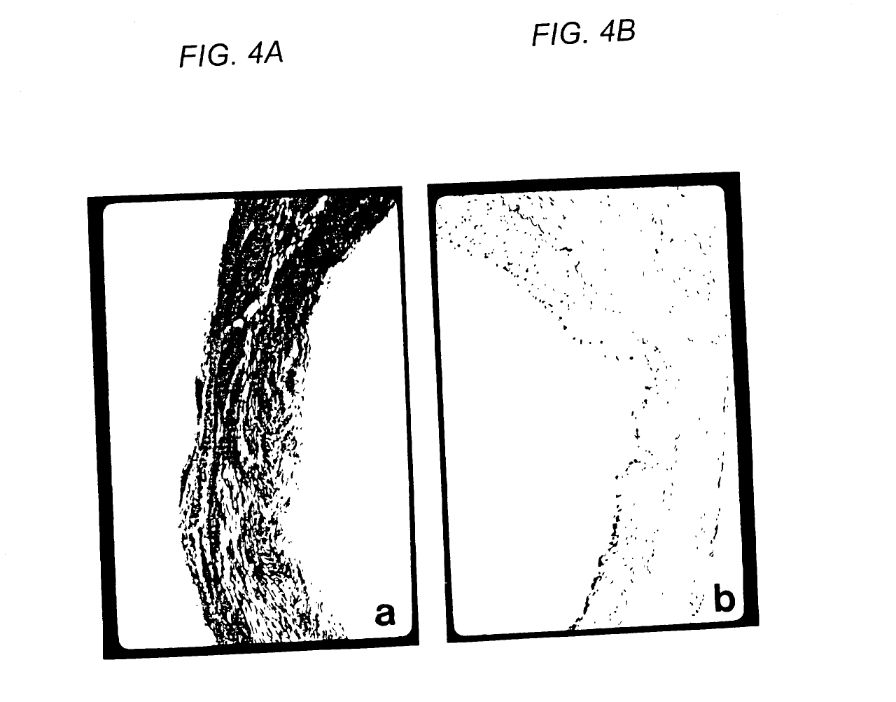

Coronary artery segments from 4 control patients exhibited no intimal thickening or inflammation as demonstrated by H&E staining (FIGS. 4A-4B). Specifically, macrophages, smooth muscle cells, foam cells or lymphocytes were not present in the intima and no cells were immunoreactive with either anti-CD40L mAb used in this study. CD40 immunoreactivity was present and confined to endothelial cells lining the vascular lumen of the control arteries (FIG. 4B). VCAM-1 or activated NF-kB was not expressed in the control vessels and ICAM-1 was weakly expressed on rare vascular endothelial cells.

Histology of Native CA and TCAD

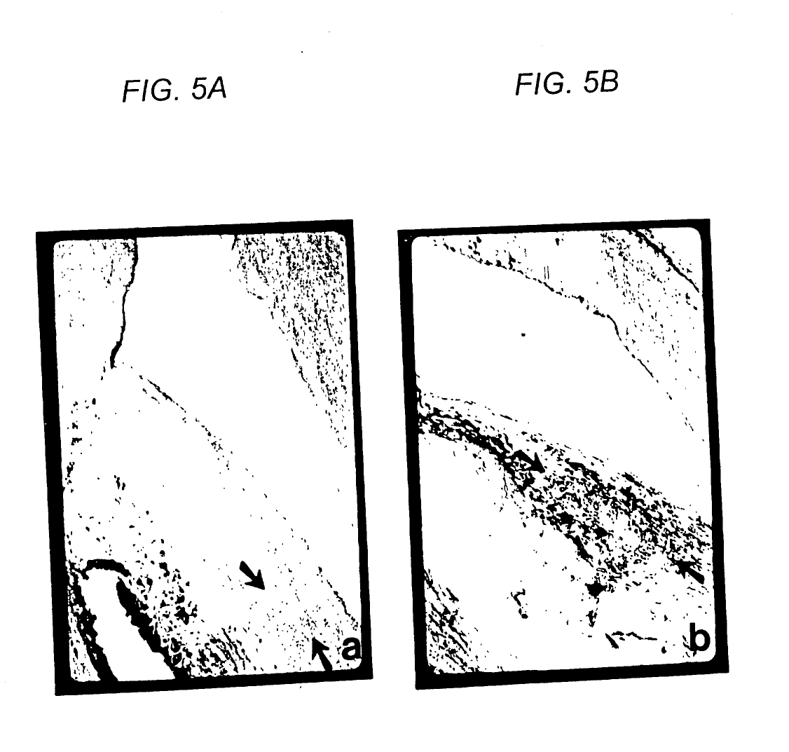

In 7 of the 10 patients with CA, coronary artery segments revealed prominent fibroatheromatous plaques with eccentric narrowing, acellular lipid-rich cores, cholesterol clefts and overlying fibrous caps. Cellularity of lesions was greatest at the “shoulder” regions which contained macrophages and lymphocytes (FIG. 5A). There were also scattered smooth muscle cells, macrophages, foam cells and foci of neovascularization in the intimal lesions. Plaques from 3 patients with mild, early vascular lesions were eccentric, small, rich in macrophages, “foam” cells and lymphocytes.

Coronary artery lesions in the 9 patients with TCAD exhibited circumferential thickening of the intima with marked narrowing of the lumen. (Table 2).

TABLE 2
Semiquantitative evaluation (scale 0-4) of cell
composition in intimal lesion of native coronary
atherosclerosis (CA) and transplant coronary artery
disease (TCAD) and the immunoreactivity for CD40. ICAM-1,
and VCAM-1. Values are expressed as mean + standard
deviation.

| | Control | | |
|---|---|---|---|
| Intimal Plaque | (n = 4) | CA (n = 10) | TCAD (n = 9) |
| Thickness | 0.3 ± 0.5 | 2.1 ± 0.9* | 3.1 ± 0.8* |
| CD4+ Lymphocytes | 0 | 1.3 ± 0.9* | 3.2 ± 0.8* |
| CD8+ Lymphocytes | 0 | 0.3 ± 0.5 | 2.6 ± 1.1* |
| Macrophages (CD68) | 0.5 ± 0.6 | 2.1 ± 0.8* | 3.8 ± 0.4* |
| Foam Cells | 0 | 1.2 ± 0.8* | 2.4 ± 1.3* |
| Smooth Muscle | 0.8 ± 1 | 1.7 ± 0.7 | 2.9 ± 0.8* |
| Cells | | | |
| Neovascularization | 0 | 1.8 ± 0.7* | 2.6 ± 0.9* |
| CD40 | 0.5 ± 0.6 | 2.2 ± 0.7* | 3.3 ± 0.9* |
| ICAM-1 | 0.5 ± 0.6 | 2.3 ± 1.7* | 3.6 ± 0.7* |
| VCAM-1 | 0.3 ± 0.5 | 1.7 ± 0.7* | 2.9 ± 0.9* |

*p, 0.05 for CA or TCAD vs. controls by Kruskal - Wallis test

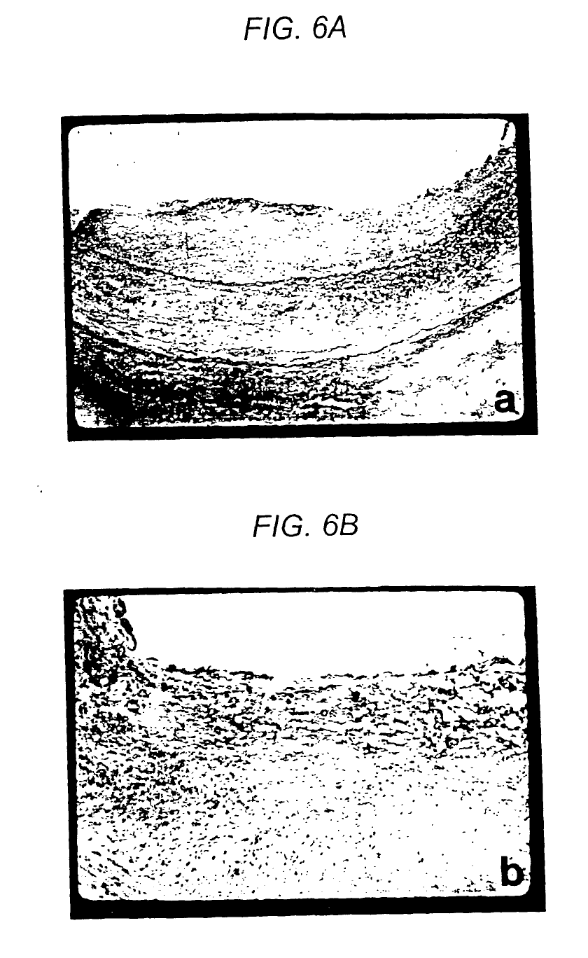

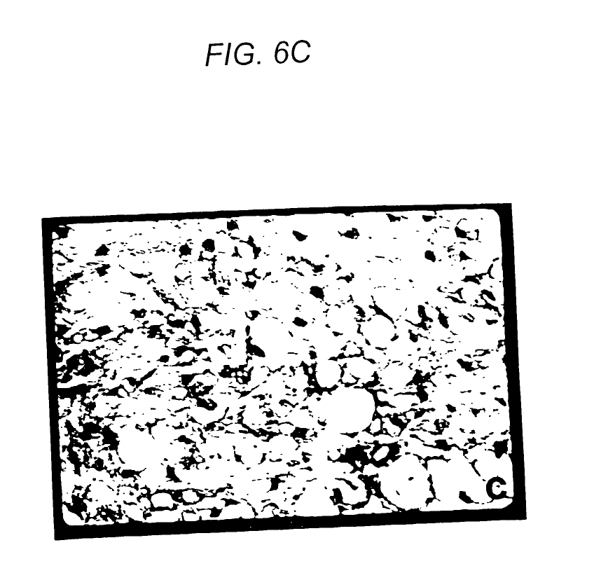

The lesions were composed of concentric layers of smooth muscle cells and interstitial matrix and there was an abundant infiltration with macrophages and lymphocytes along with areas of neovascularization. In 4 coronary arteries lipid-rich atheromatous lesions and “foam” cells were discerned in addition to the concentric layers of smooth muscle cells (FIGS. 6A-C). Subendothelial collections of lymphocytes (“endothelitis”) and aggregates of lymphocytes in the adventitia were also features noted in TCAD lesions.

Immunohistochemical Analysis of CD40L Expression in CA and TCAD

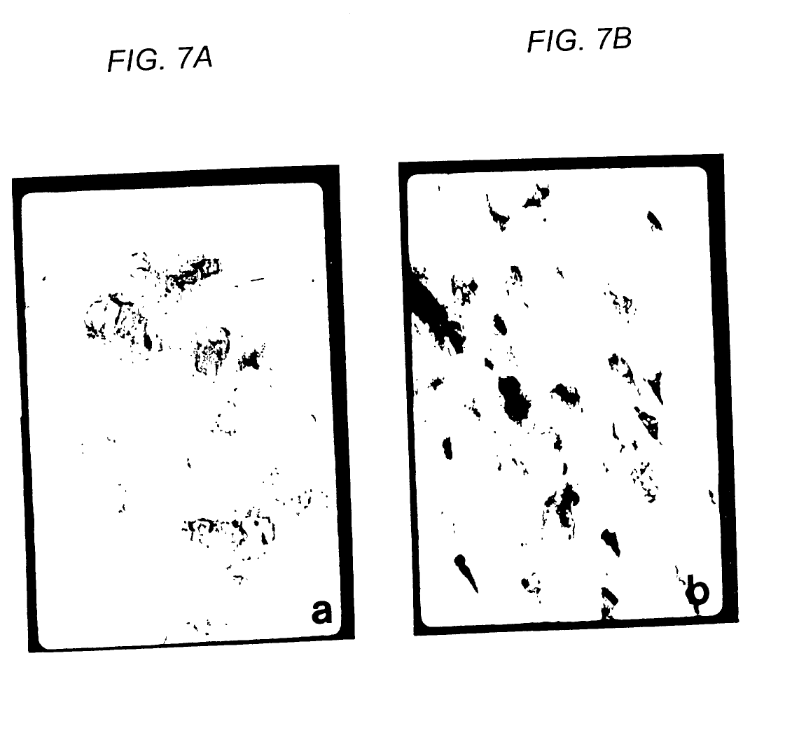

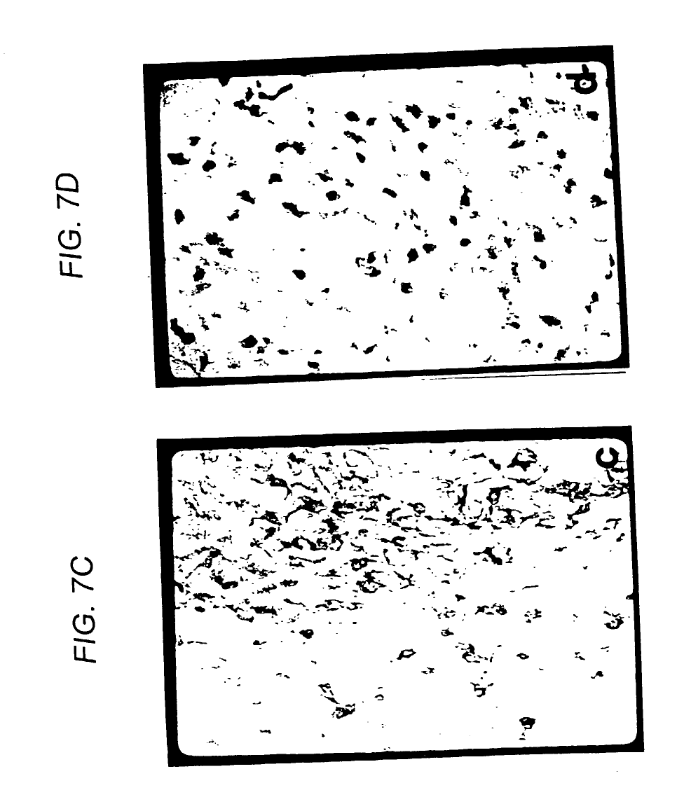

In marked contrast to normal coronary arteries, which are devoid of infiltrating lymphocytes or CD40L expressing cells, both CA and TCAD lesions contained CD40L⁺ cells. In native atherosclerosis positive immunostaining for CD40L was confined to a minority of intimal lymphocytes. CD40L staining was usually weak and observed either in small cytoplasmic granules or on the surface of cells (FIGS. 7A-D). In native CA most of the intimal lymphocytes were CD4⁺ T cells; only rare CD8+ T cells were present (FIGS. 7A-D). Analysis of serial sections stained with anti-CD4 or anti-CD8 mAbs suggest that the CD40L+ lymphocytes were primarily CD4+ T cells. Endothelial cells, smooth muscle cells, macrophages and “foam” cells did not react with either anti-CD40L mAb used in this study. No staining was noted with isotype control mAbs.

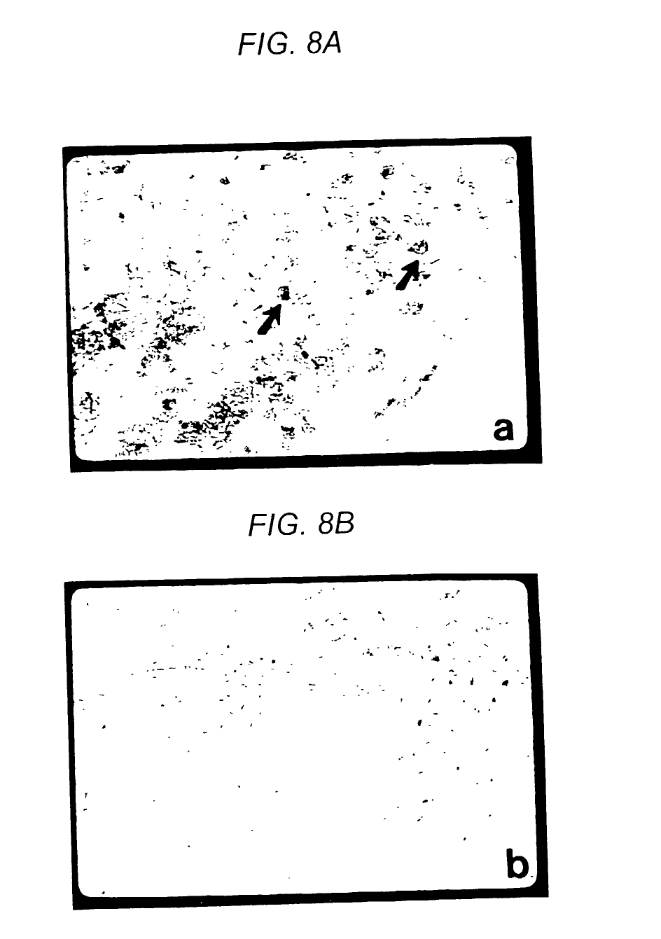

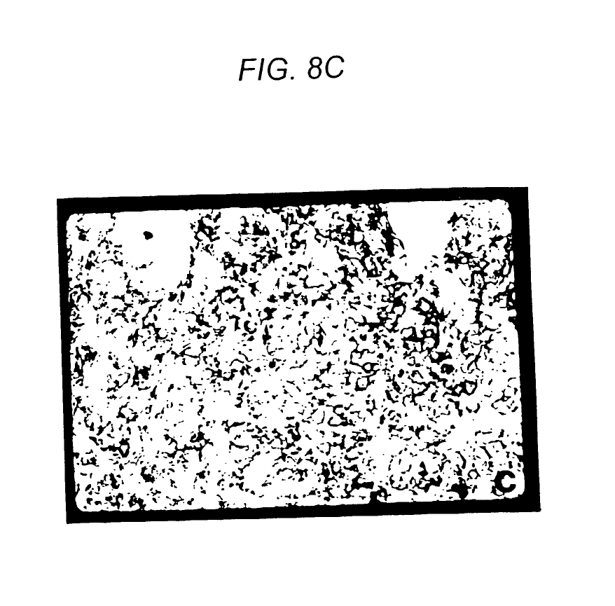

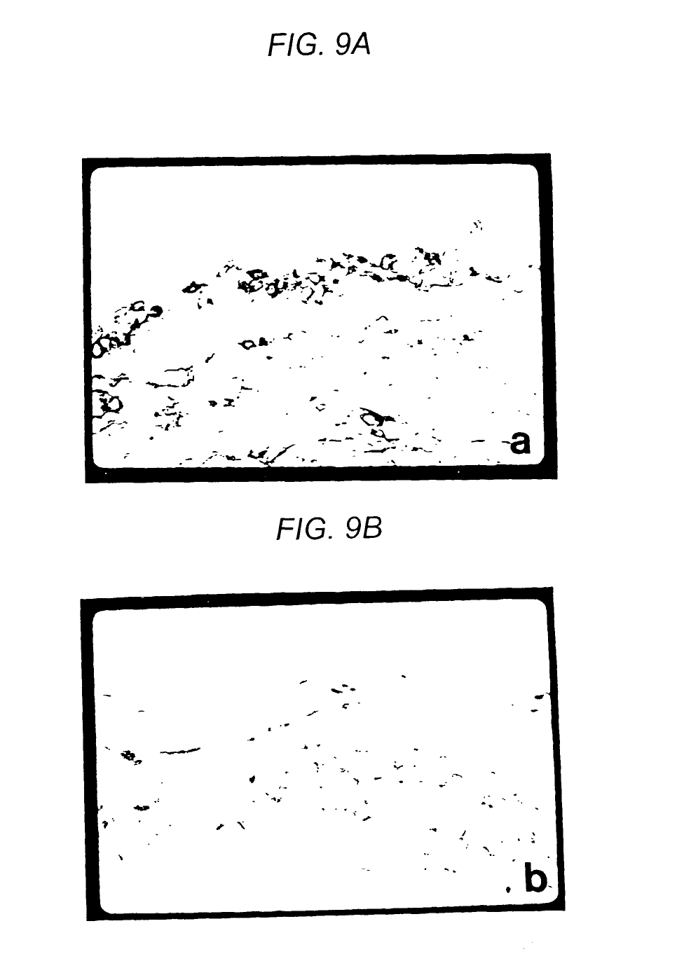

In TCAD lesions, positive immunostaining for CD40L was also exclusively associated with lymphocytes (FIGS. 8A-C). In contrast to CA, both CD8+ and CD4+ T cells were present in TCAD lesions. However, CD8+ T cells were predominately found in subendothelial areas of “endothelitis” (FIGS. 9A-B) while CD4+ T cells localized in aggregates deep in the intima adjacent to the internal elastic membrane (FIGS. 8A-C) and adventitia of coronary arteries. The expression of CD40L correlated spatially with CD4+ T cells in the intima and adventitia of coronary arteries with TCAD. The number of CD40L+ T cells was higher in TCAD than in native CA lesions. Similar to CA, endothelial cells, smooth muscle cells, macrophages or “foam” cells in TCAD lesions did not react with either anti-CD40L mAb used in this study (FIGS. 9A-B). These data indicate that CD40L expressing cells, probably CD4+ T cells, are present in the lesions of native CA and TCAD.

Immunohistochemical Analysis of CD40 Expression in CA and TCAD

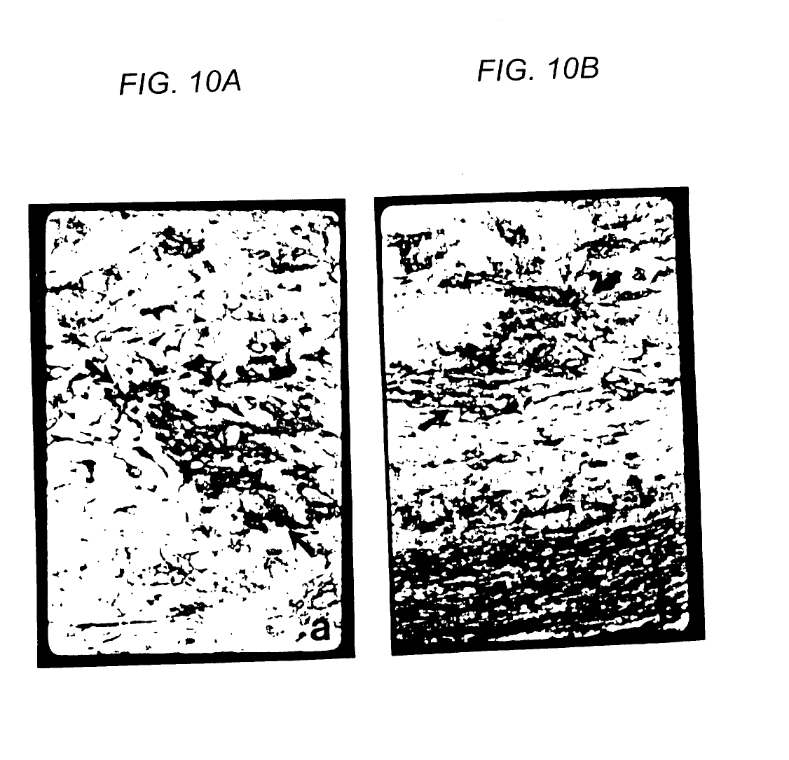

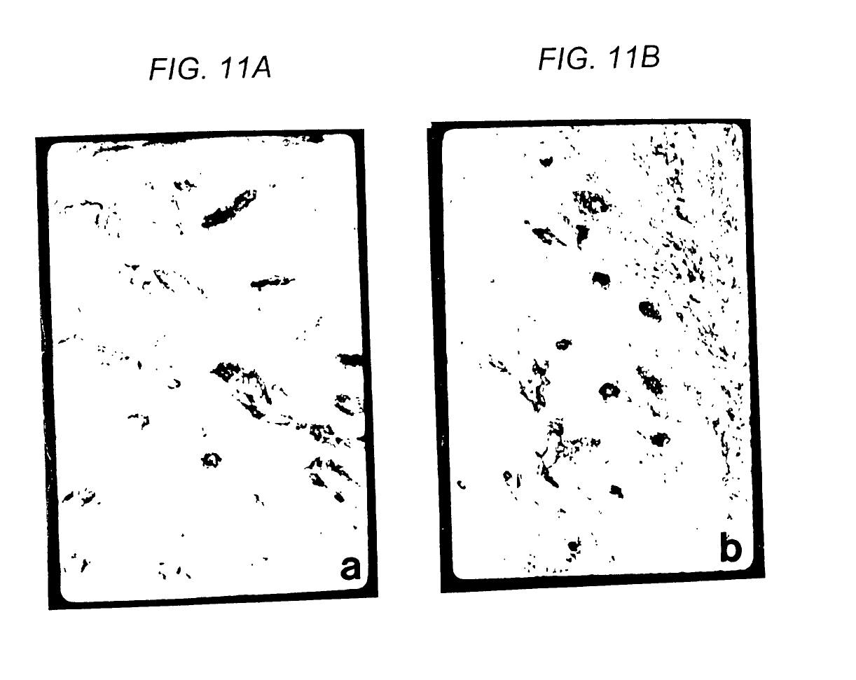

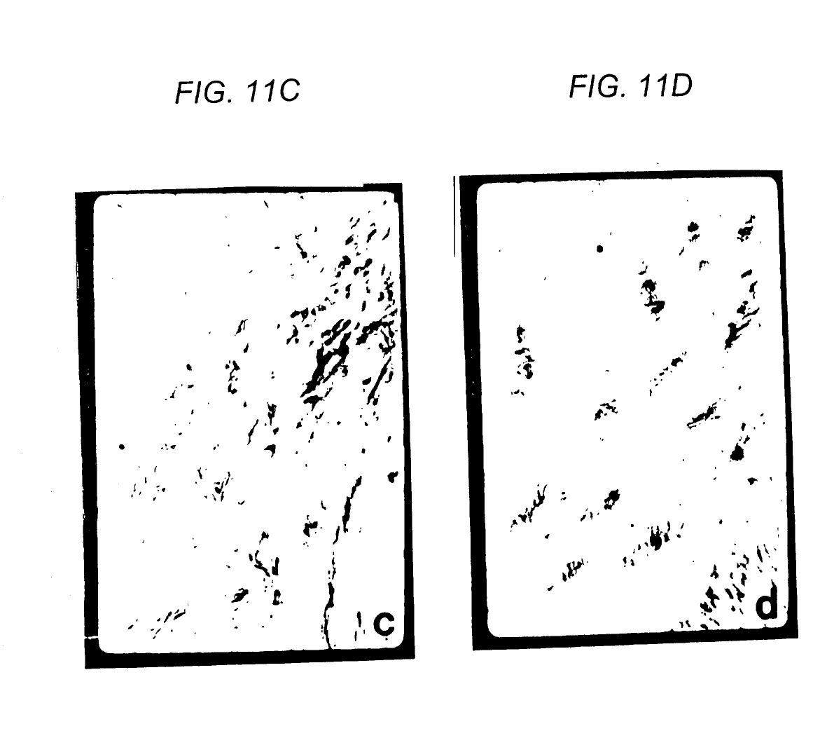

In contrast to the weak CD40 expression limited to luminal endothelial cells in normal coronary arteries (FIGS. 4A-B), CD40 immunoreactivity was upregulated and broadly distributed in the lesions of native CA (FIGS. 5A-B). CD40 expression was noted on endothelial cells, smooth muscle cells, macrophages and “foam” cells. There was a significantly higher mean number of CD40 positive cells in intimal lesions of native CA than in control arteries (2.2+0.7 versus 0.5+0.6, Table 2). Dual immunostaining with macrophage or smooth muscle cell specific markers confirmed that these cells and “foam” cells of both lineages express CD40 (FIGS. 10A-B). Interestingly, CD40+ smooth muscle cells were present in the intima near inflammatory infiltrates, whereas smooth muscle cells in the arterial media did not show positive immunoreactivity for CD40 (FIGS. 10A-B). Analysis of serial sections stained with CD40 or the endothelial marker CD34 suggested that endothelial cells lining the intimal neovessels and adventitial vasa vasorum were also strongly CD40+ (FIGS. 11A-D).

In arteries from patients with TCAD, the pattern of distribution of CD40 expression was similar to native CA. However, the average score for CD40 immunoreactivity was significantly higher in TCAD than in native CA or control arteries (Table 2). Double immunostaining indicated that intimal smooth muscle cells and macrophages express CD40 (FIGS. 10A-B). Moreover, foam cells (FIGS. 6A-B) and endothelial cells lining the vascular lumen, intimal neovessels and adventitial vasa vasorum were markedly CD40+. Together, these data demonstrate that endothelial cells, smooth muscle cells and macrophages express CD40 in both native CA and TCAD.

Relationship of CD40 Expression to Intercellular Adhesion Molecules and Activation of NF-kB in CA and TCAD Lesions.

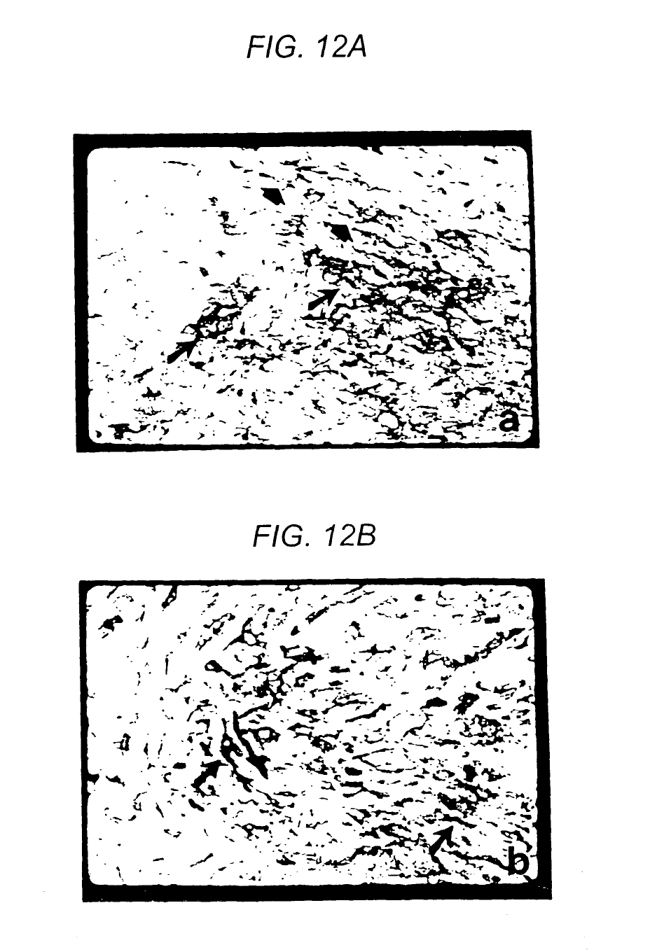

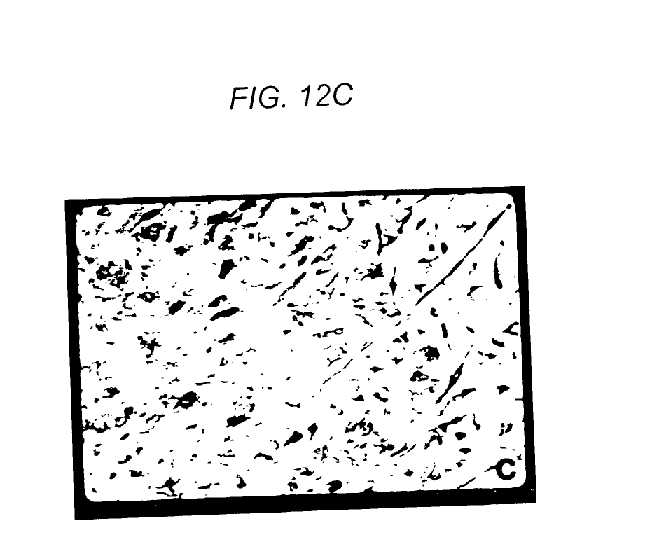

Macrophages and endothelial cells in CA and TCAD express intercellular adhesion molecules that regulate the trafficking of leukocytes into the lesion. Because ligation of CD40 induces upregulation of intercellular adhesion molecules and activation of NF-κB on cells in vitro, it was then asked if CD40 expression was associated with the co-expression of intercellular adhesion molecules or NF-kB in CA or TCAD lesions. First it was demonstrated in native CA that luminal endothelial cells manifested focal positive immunostaining for ICAM-1 with rare endothelial cells expressing VCAM-1. In contrast, endothelial cells lining intimal neovessels and adventitial vasa vasorum were strongly positive for ICAM-1 and VCAM-1 (FIGS. 11A-D). Intimal smooth muscle cells, macrophages and “foam” cells were also moderately to strongly positive for ICAM-1 and VCAM-1 (FIGS. 12A-C). There was a significant correlation (p<0.05) between CD40 scores and those for ICAM-1 (r=0.85) and VCAM-1 (r=0.72). The number of intimal lymphocytes correlated significantly with the scores for CD40 and the leukocyte adhesion molecules (Table 3).

TABLE 3
Correlation of scores (0-4) for various cell
types of the intimal lesions of CA (n = 10) or TCAD (n = 9)
with scores (0-4) for expression of CD40 and adhesion
molecules (ICAM-1, VCAM-1). Values are expressed as the
Spearmen correlation coefficient (range −1 to 1, with “0”
no correlation and “−1” or “1” perfect correlation)

| Cell Type | Group | CD40 | ICAM-1 | VCAM-1 |
|---|---|---|---|---|
| T-lymphocytes | CA | 0.78* | 0.77* | 0.83** |
| (CD4+ & CD8+) | TCAD | 0.79* | 0.87** | 0.77* |
| Macrophages | CA | 0.93*** | 0.84** | 0.77* |
| (CD68+) | TCAD | 0.81** | 0.68* | 0.55 |
| Foam Cells | CA | 0.81** | 0.68* | 0.36 |
| | TCAD | 0.44 | 0.33 | 0.26 |
| Smooth Muscle | CA | 0.72* | 0.81** | 0.56 |
| Cells (SMA+) | TCAD | 0.12 | 0.38 | 0.02 |
| Neovessels | CA | 0.69* | 0.72* | 0.53 |
| (CD34+) | TCAD | 0.85** | 0.87** | 0.77* |

*p < 0.05, **p < 0.01 and ***p < 0.0001 level of significance for Spearman Correlation.

Of all listed cell types only the score for intimal lymphocytes correlated significantly with CD40 expression and extent of ICAM-1 and VCAM-1 in intimal plaques in both CA and TCAD suggesting that lymphocytes are involved in the induction of CD40 and adhesion molecules in both diseases. Macrophages and neovascularization also showed significant correlation with CD40 expression in CA and TCAD.

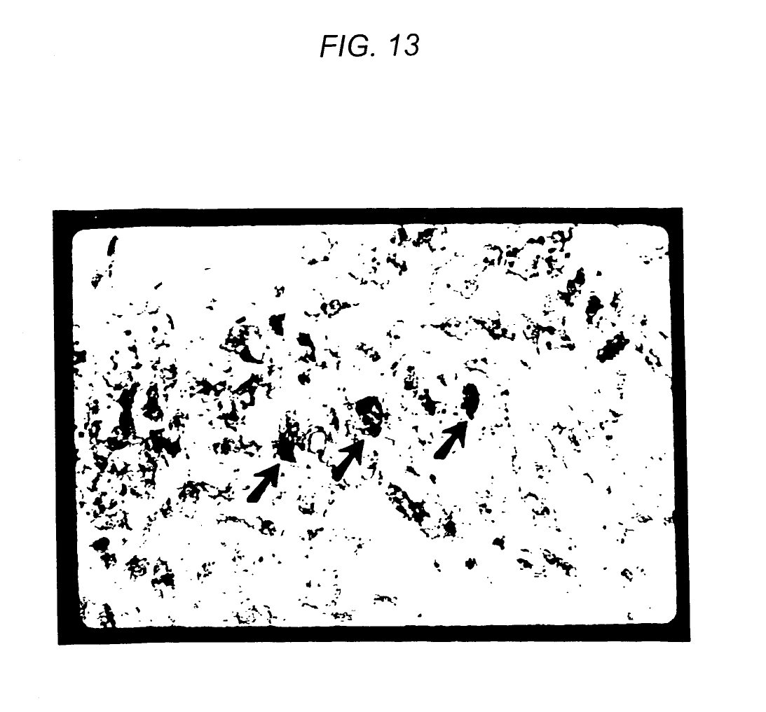

Double immunostaining of CA lesions with anti-CD40 mAb and anti-ICAM-1 mAb or anti-VCAM-1 mAb showed that CD40 colocalized with these adhesion molecules on many cells (FIGS. 12A-C). In addition, activated NF-κB (FIG. 13) was observed in the nuclei of neointimal endothelial cells, macrophages and smooth muscle cells and dual immmunolabeling demonstrated that many CD40+ cells also expressed activated NF-κB.

In TCAD, strongly positive immunostaining for ICAM-1 and VCAM-1 was present on luminal endothelial cells, particularly those near foci of endothelitis. Endothelial cells of intimal neovessels adventitial vasa vasorum were strongly immunoreactive for ICAM-1 and VCAM-1. Scores for immunostaining of the adhesion molecules in TCAD were higher than in CA or normal coronary arteries (Table 2). There was a significant correlation (p<0.05) between CD40 scores and those for ICAM-1 (r=0.82) and VCAM-1 (r=0.89). The number of intimal lymphocytes also correlated significantly with the expression of CD40, ICAM-1 and VCAM-1 (Table 3). Similar to CA, two-color immunohistochemistry studies demonstrated that many CD40+ cells in TCAD lesions co-express ICAM-1 or VCAM-1 (FIGS. 12A-C). Immunostaining for the activated nuclear form of NF-κB was more widely distributed in TCAD than in native CA. NF-kB positive macrophages and smooth muscle cells were consistently CD40+ (FIG. 13). Together, these studies demonstrate that in lesions of both native CA and TCAD, CD40 is coexpressed on many cells with intercellular adhesion molecules and/or NF-κB.

Discussion

Native atherosclerosis (CA) and transplant related atherosclerosis (TCAD) are inflammatory diseases mediated by complex interactions between activated T cells, endothelial cells, macrophages and smooth muscle cells (2, 8, 12, 13, 17). T cells are thought to play roles in the pathogenesis of CA and TCAD, however the mechanisms by which they participate in these processes are not fully known (5, 9, 50). Studies have shown that CD40L, an activation induced CD4+ T cell surface molecule, delivers contact-dependent activation signals to CD40 expressing endothelial cells and macrophages that result in the production of pro-inflammatory molecules, such as intercellular adhesion molecules ICAM-1 and VCAM-1 (31, 32, 35-37) and the activation of the transcriptional activating factor NF-κB (43-45, in vitro). Interestingly, TCAD in murine models is at least partly dependent on CD40L-CD40 interactions (51). In the study by Larson and colleagues, anti-CD40L mAb therapy markedly inhibited allogenic hetertopic transplant rejection and partially blocked the associated vasculopathy. Moreover, TCAD in this model was almost completely prevented by administering the combination of anti-CD40L mAb and CTLA4-Ig fusion protein, a molecule that blocks T cell costimulatory pathways (51). It is possible that CD40L-CD40 interactions may participate in the pathogenesis of CA and/or TCAD in humans.

To investigate this hypothesis further immunohistochemical techniques were applied to normal and atherosclerotic coronary arteries to study the expression and cellular distribution of CD40L and CD40. Normal coronary arteries do not contain CD40L expressing cells and CD40 immunoreactivity was restricted to luminal endothelial cells in these vessels. In contrast, CD40L is expressed on lymphocytes in lesions of both native CA and TCAD. It was found that CA lesions contained few CD8+ T cells while TCAD lesions contained CD8+ T cells in close proximity to the luminal endothelium (“endothelitis”) and CD4+ T cells deeper in the intima and adventitia. Based on localization and staining of serial sections with anti-CD4 mAb or anti-CD8 mAb, it was concluded that CD40L+ lymphocytes are most likely CD4+ T cells in the lesions of both diseases. Utilizing two different anti-CD40L mAb it was found that CD40L immunoreactivity was weak and either granular and cytoplasmic or cell surface associated. A similar pattern of CD40L immunoreactivity was noted in a study of CD40L and CD40 expression in glomerulonephritis (46). The weak and frequent cytoplasmic staining pattern of CD40L expression in inflammatory tissues may be related to the transient nature of CD40L expression on activated T cells (27-29) and the fact that engagement of CD40 on target cells induces rapid down-modulation of CD40L by receptor-mediated endocytosis (52) and shedding (53). These regulatory mechanisms probably serve to focus CD40L mediated signaling events to appropriate cognate target cells.

It was found that CD40 expression was markedly upregulated on many cells in the lesions of both diseases. Macrophages and “foam” cells expressing CD40 were particularly prominent in the inflammatory infiltrate of the “shoulder” regions of lipid-rich plaques, which are known to contain dense inflammatory infiltrates (54, 55). CD40 expression was also upregulated on luminal endothelial cells in both diseases and this was particularly prominent in TCAD. Intimal neovessel and adventitial vasa vasorum endothelial cells in both diseases were strongly CD40+. CD40 expressing smooth muscle cells were present in the intima of both CA and TCAD, usually in close proximity to inflammatory infiltrates. Interestingly, smooth muscle cells in the media of the same vessels were CD40−. IFN-γ upregulates CD40 expression on many cells in vitro (33, 35-37, 56) including smooth muscle cells, and this effect is enhanced by cytokines such as IL-1β and TNF-α (36).

Therefore, the marked upregulation of CD40 expression on many cell types in these lesions may be a consequence of cytokine release by lesional T cells, macrophages and other cells. Double immunostaining indicated that many CD40+ cells also co-express intercellular adhesion molecules ICAM-1 and VCAM-1, as well as, the activated form of NF-κB. Together, the current study demonstrates the presence of CD40L+ T cells and activated CD40+ target cells in the vascular lesions of native CA and TCAD.

Early studies showed that CD40 was expressed on some epithelial cell tumors and B cells (57, 58). More recently it has been noted that CD40 is constitutively expressed or inducible on many cell types in vitro (33-37, 56). Furthermore, it is becoming increasingly evident that CD40L-CD40 interactions play key roles in cell-mediated inflammatory reactions in vivo (31, 32). In this regard, recent reports demonstrate in situ CD40L and/or CD40 expression in human inflammatory diseases (35, 46, 59). For example, CD40 expression is upregulated on macrophages infiltrating the brains of patients with multiple sclerosis (59), on dermal endothelial cells and keratinocytes in psoriasis (35), and on many cells in the kidneys of patients with inflammatory glomerulonephritides (46). Moreover, inflammatory infiltrates in the brains of patients with multiple sclerosis (59) and in the kidneys of patients with inflammatory glomerulonephritides 46 contain CD40L+ T cells. It is therefore likely that CD40 expression is upregulated in many inflammatory diseases and represents a molecular mechanism that permits T cells to deliver pro-inflammatory signals to a wide variety of target cells. In this regard, the findings presented herein that CD40 expression is upregulated in CA and TCAD, and that CD40L+ infiltrating T cells are found in lesions, serves as evidence of the hypothesis that immune mediated inflammatory reactions play roles in the pathogenesis of these diseases (5-7, 9, 18, 21, 23, 50).

Observations regarding CD40L mediated activation of endothelial cells and macrophages in vitro and studies of CD40L-CD40 interactions in the pathogenesis of murine models of TCAD, suggest possible pathogenic roles for CD40L-CD40 interactions in CA and TCAD. For example, CD40L mediated signals upregulate ICAM-1 and VCAM-1 expression on endothelial cells, in vitro (35-37). These intercellular adhesion molecules, which regulate the egress and retention of leukocytes in inflammatory sites, are upregulated on endothelial cells in CA and TCAD and are particularly prominent on intimal neovessel and vasa vasorum endothelial cells (49, 60). Therefore, it is of interest that many CD40+ cells were found in CA and TCAD lesions, and in particular intimal and vasa vasorum endothelial cells, co-express ICAM-1 and/or VCAM-1. Upregulation of ICAM-1 and VCAM-1 is known to be dependent on activation of NF-kB (61). In the present study it was also demonstrated that CD40+ intimal macrophages, smooth muscle cells and endothelial cells express the activated form of NF-kB. These studies suggest that CD40L+ CD4+ T cells may induce upregulation of intercellular adhesion molecules on CD40+ target cells in CA and TCAD, possibly in part by activating NF-kB.

CD40L mediated signals also induce endothelial cells to secrete IL-6 and IL-8 (62) and promotes a procoagulant surface by upregulating tissue factor and down-regulating thrombomodulin expression. With regard to macrophages, CD40L-CD40 interactions induce these cells to secrete proinflammatory cytokines (IL-1α, IL-1β, IL-6 and TNF-α), chemokines, matrix metalloproteinases and express tissue factor in vitro (33, 34, 38, 41, 42). All these pro-inflammatory molecules probably play roles in the pathogenesis of CA and TCAD (10, 17, 63-66). Ligation of CD40 on macrophages also induces NO production (39, 40).

Interestingly, blocking CD40L-CD40 interactions in murine models of TCAD is associated with down-regulation of iNOS expression and reduction of TCAD lesions (51). It was demonstrated that iNOS is expressed in the lesions of CA (67, 68), cardiac allograft rejection (69, 70) and TCAD (71, 72). CD40L mediated signals may be involved in promoting the production of any of these molecules in CA or TCAD. CD40L-CD40 interactions clearly have pro-inflammatory effects in murine models of TCAD (51), as well as, collagen-induce arthritis (73), lupus-like glomerulonephritis (74) and experimental allergic encephalomyelitis (59).

An investigation (62) of the expression of CD40L and CD40 in human carotid atherosclerosis was carried out. It was found that CD40 was upregulated in lesions and had a broad cellular distribution. CD40L was reported to be widely expressed on smooth muscle cells, endothelial cells and macrophages in the atherosclerotic lesions, whereas in the present study using two different anti-CD40L mAbs, CD40L expression was restricted to T cells. Herein, in situ CD40L expression on macrophages, endothelial cells or smooth muscle cells in either disease was not observed. Similarly, it was found that CD40L immunoreactivity confined to T cells in other inflammatory diseases, including glomerulonephritis (46), rheumatoid arthritis and chronic sinusitis. Additionally, Gerritse et. al. reported that CD40L expression was restricted to CD4+ T cells in multiple sclerosis plaques (59). Discrepancies between results herein and those of Mach and colleagues are currently unclear but may relate to subtle differences in immunohistochemical techniques or in the nature of the lesions.

References

1. Nilsson, J. 1993. Transplant Proc. 25:2063-2064.

2. Munro, J., and R. Cotran. 1988. Lab Invest. 58:249-261.

3. Cramer, D., et al. 1992. J Heart Lung Transplant. 11:458-466.

4. Billingham, M. 1992. J Heart Lung Transplant. 11:S38-S44.

5. Zhou, X., et al. 1996. Am J Pathol. 149:359-366.

6. Wick, G., et al. 1995. Immunol. Today. 16:27-33.

7. Stemme, S., et al. 1992. Arteriosclerosis and Thrombo. 12:206-211.

8. Ross, R. 1993. Nature. 362:801-809.

9. Lichtman, A., et al. 1996. Am J Pathol. 149:351-357.

10. Kishikawa, H., et al. 1993. Virchows Arch. 423:433-442.

11. Krensky, A. 1994. Kidney Int. 45:50S-56S.

12. Salomon, R., et al. 1991. Am J Pathol. 138:791-798.

13. Shi, C., et al. 1996. Proc Natl Acad Sci, USA. 93:4051-4056.

14. Jonasson, L., et al. 1985. J Clin Invest. 76:125-131.

15. Russell, P. S., et al. 1994. Am. J. Pathol. 144:260-274.

16. Moyer, C., et al. 1992. J Pathol. 138:951-960.

17. Libby, P., and Z. Gallis. 1995. Ann NY Acad Sci. 748:158-168.

18. Stemme, S., et al. 1995. Proc Natl Acad Sci, USA. 92:3893-3897.

19. Witztum, J., and D. Steinberg. 1991. J Clin Invest. 88:1785-1792.

20. de Lorgeril, M., et al. 1993. Am Heart J. 125:974-980.

21. Emeson, E., et al. 1996. Am J Pathol. 149:675-685.

22. Russell, P., et al. 1994. Transplantation. 57:1367-1371.

23. Russell, M., et al. 1996. J Clin Invest. 97:833-838.

24. Hancock, W., et al. 1996. Proc Natl Acad Sci. 93:13967-13972.

25. Graf, D., et al. 1992. Eur J Immunol. 22:3191-3194.

26. Armitage, R. J., et al. 1992. Nature. 357 (6373) :80-2.

27. Lane, P., et al. 1992. Eur J Immunol. 22:2573-2578.

28. Lederman, S., et al. 1992. J Exp Med. 175 (4):1091-101.

29. Noelle, R. et al. 1992. Proc Natl Acad Sci USA. 89:6550-6554.

30. Banchereau, J., et al. 1994. Annu. Rev. Immunol. 12:881-922.

31. Noelle, R. 1996. Immunity. 4:415-419.

32. Stout, R., and J. Suttles. 1996. Immunol Today. 17:487-492.

33. Alderson, M. R., et al. 1993. J Exp Med. 178 (2):669-74.

34. Caux, C., et al. 1994. J. Exp. Med. 180:1263-1272.

35. Hollenbaugh, D., et al. 1995. J. Exp. Med. 182:33-40.

36. Karmann, K., et al. 1995. Proc Natl Acad Sci, USA. 92:4342-4346.

37. Yellin, M. J., et al. 1995. J. Exp. Med. 182:1857-1864.

38. Kiener, P., et al. 1995. J Immunol. 155:4917-4925.

39. Stout, R., et al. 1996. J Immunol. 156:8-11.

40. Tian, L., et al. 1995. Eur J Immunol. 25:306-309.

41. Pradier, O., et al. 1996. Eur J Immunol. 26:3048-3054.

42. Malik, N., et al. 1996. J Immunol. 156:3952-3960.

43. Berberich, I., et al. 1994. J Immunol. 153:4357-4366.

44. Hess, S., et al. 1995. J Immunol. 155:4588-4595.

45. Karmann, K., et al. 1996. J Exp Med. 184:173-182.

46. Yellin, M., et al. 1997. Arthritis Rheum. 40:124-134.

47. Brand, K., et al. 1996. J Clin Invest. 97:1715-1722.

48. Kuamamoto, M., et al. 1995. Human Path. 26:450-456.

49. O'Brien, K., et al. 1996. Circulation. 93: 672-682.

50. Haraoka, S., et al. 1995. Virchows Arch. 426:307-315.

51. Larsen, C., et al. 1996. Nature. 381:434-438.

52. Yellin, M. J., et al. 1994. J Immunol. 152 (2):598-608.

53. Graf, D., et al. 1995. Eur J Immunol. 25:1749-1754.

54. Bjoerkerud, S., and B. Bjoerkerud. 1996. Am J Pathol. 149:367-380.

55. van der Wal, A., et al. 1994. Circulation. 89:36-44.

56. Yellin, M. J., et al. 1995. J. Leuk. Biol. 58:209-216.

57. Pauli, S., et al. 1985. Cancer Immunol. Immunother. 20:23-28.

58. Clark, E. A., and J. A. Ledbetter. 1986. Proc. Natl. Acad. Sci. USA. 83:4494-4498.

59. Gerritse, K., et al. 1996. Proc. Natl. Acad. Sci. 93:2499-2504.

60. Davies, M., et al. 1993. J Pathol. 171:223-229.

61. Collins, T., et al. 1995. FASEB J. 9:899-909.

62. Mach, F., et al. 1997. Proc Natl Acad Sci, USA. 94:1931-1936.

63. Berliner, J., et al. 1995. Circulation. 91:2488-2496.

64. Libby, P., et al. 1995. J Cardiovasc Pharm. 25 Suppl 2:S9-12.

65. Li, Z., et al. 1996. Am J Path. 148:121-128.

66. Galis, Z., et al. 1994. J Clin Invest. 94:2493-2503.

67. Buttery, L., et al. 1996. Lab. Invest. 75:77-85.

68. Aji, W., et al. 1997. Circulation. 95:430-437.

69. Yang, X., et al. 1994. J Clin Invest. 94:714-721.

70. Worrall, N., et al. 1995. J Exp Med. 181:63-70.

71. Russell, M., et al. 1995. Circulation. 92:457-464.

72. Akyurek, L., et al. 1996. Am J Pathol. 149:1891-1990.

73. Durie, F. H., et al. 1993. Science. 261:1328-1330.

74. Mohan, C., et al. 1995. J Immunol. 154:1470-1480.

What is claimed is:

1. A method of inhibiting activation by CD40 ligand of smooth muscle cells bearing CD40 on the surface of the cells, comprising contacting the cells with an agent capable of inhibiting interaction between CD40 ligand and CD40 on the cells, the agent being present in an amount effective to inhibit activation of the cells.

2. The method of claim 1, wherein the smooth muscle cells are smooth muscle cells of the bladder, vascular smooth muscle cells, aortic smooth muscle cells, coronary smooth muscle cells, pulmonary smooth muscle cells, or gastrointestinal smooth muscle cells.

3. The method of claim 2, wherein the gastrointestinal smooth muscle cells are esophageal smooth muscle cells, stomachic smooth muscle cells, smooth muscle cells of the small intestine, or smooth muscle cells of the large intestine.

4. The method of claim 1, wherein the agent inhibits binding of CD40 ligand to CD40 on the cells.

5. The method of claim 1, wherein the agent is a protein.

6. The method of claim 5, wherein the protein comprises an antibody or portion thereof.

7. The method of claim 6, wherein the antibody is a monoclonal antibody.

8. The method of claim 7, wherein the monoclonal antibody specifically binds to the epitope to which monoclonal antibody 5c8 (ATCC Accession No. HB 10916) specifically binds.

9. The method of claim 8, wherein the monoclonal antibody is monoclonal antibody 5c8 (ATCC Accession No. HB 10916).

10. The method of claim 7, wherein the monoclonal antibody specifically binds to CD40.

11. The method of claim 10, wherein the antibody is humanized, chimeric, or primatized.

12. The method of claim 7, wherein the monoclonal antibody is a chimeric antibody.

13. The method of claim 7, wherein the monoclonal antibody is a humanized antibody.

14. The method of claim 6, wherein the portion of the antibody comprises a complementarity determining region or variable region of a light or heavy chain.

15. The method of claim 6, wherein the portion of the antibody comprises a complementarity determining region or a variable region.

16. The method of claim 15, wherein the portion of the antibody comprises a Fab or a single chain antibody.

17. The method of claim 5, wherein the protein comprises soluble extracellular region of CD40 ligand, or variant thereof including conservative substituents, or portion thereof; or soluble extracellular region of CD40, or variant thereof including conservative substituents, or portion thereof.

18. The method of claim 17, wherein the soluble extracellular region of CD40 ligand or CD40 is a monomer.

19. The method of claim 17, wherein the soluble extracellular region of CD40 is an oligomer.

20. The method of claim 17, wherein the protein comprising soluble extracellular region of CD40 or portion thereof or CD40 ligand or portion thereof further comprises an Fc region fused to the extracellular region of CD40 or portion thereof or CD40 ligand or portion thereof.

21. The method of claim 20, wherein the Fc region is capable of binding to protein A or protein G.

22. The method of claim 21, wherein the Fc region comprises IgG, IgA, IgM, IgD, or IgE, or subclasses thereof.

23. The method of claim 22, wherein:
the IgG is IgG₁, IgG₂, IgG₃, or IgG₄; or
the IgA is IgA₁ or IgA₂.

24. The method of claim 1, wherein the agent is nonprotein.

25. The method of claim 1, wherein the agent is selected from a library of known agents.

26. The method of claim 1, wherein the agent is modified from a known agent.

27. The method of claim 26, wherein the modified agent is designed by structure optimization of a lead inhibitory agent based on a three-dimensional structure of a complex of soluble extracellular region of CD40 ligand or portion thereof with the lead inhibitory agent.

28. The method of claim 1, wherein the agent is selected by a screening method, which comprises:
isolating a sample of cells;
culturing the sample under conditions permitting activation of CD40-bearing cells;
contacting the sample with cells expressing a protein which is specifically recognized by monoclonal antibody 5c8 produced by the hybridoma having ATCC Accession No. HB 10916, or with a protein which is specifically recognized by monoclonal antibody 5c8 produced by the hybridoma having ATCC Accession No. HB 10916, effective to activate the CD40-bearing cells;
contacting the sample with an amount of the agent effective to inhibit activation of the CD40-bearing cells if the agent is capable of inhibiting activation of the CD40-bearing cells; and
determining whether the cells expressing the protein which is specifically recognized by monoclonal antibody 5c8 produced by the hybridoma having ATCC Accession No. HB 10916, or with the protein which is specifically recognized by monoclonal antibody 5c8 produced by the hybridoma having ATCC Accession No. HB 10916, activate the CD40-bearing cells in the presence of the agent.

29. The method of claim 28, wherein the agent is selected from a library of known agents.

30. The method of claim 29, wherein the known agents are nonprotein agents.

31. A method of inhibiting activation by CD40 ligand of smooth muscle cells bearing CD40 on the surface of the cells, in a subject, comprising administering to the subject an agent capable of inhibiting interaction between CD40 ligand and CD40 on the cells, the agent being present in an amount effective to inhibit activation of the cells in the subject.

32. The method of claim 31, wherein the smooth muscle cells are smooth muscle cells of the bladder, vascular smooth muscle cells, aortic smooth muscle cells, coronary smooth muscle cells, pulmonary smooth muscle cells, or gastrointestinal smooth muscle cells.

33. The method of claim 32, wherein the gastrointestinal smooth muscle cells are esophageal smooth muscle cells, stomachic smooth muscle cells, smooth muscle cells of the small intestine, or smooth muscle cells of the large intestine.

34. The method of claim 31, wherein the agent inhibits binding of CD40 ligand to CD40 on the cells.

35. The method of claim 31, wherein the agent is a protein.

36. The method of claim 35, wherein the protein comprises an antibody or portion thereof.

37. The method of claim 36, wherein the antibody is a monoclonal antibody.

38. The method of claim 37, wherein the monoclonal antibody specifically binds to the epitope to which monoclonal antibody 5c8 (ATCC Accession No. HB 10916) specifically binds.

39. The method of claim 38, wherein the agent is monoclonal antibody 5c8 (ATCC Accession No. HB 10916).

40. The method of claim 37, wherein the monoclonal antibody specifically binds to CD40.

41. The method of claim 40, wherein the antibody is humanized, chimeric, or primatized.

42. The method of claim 37, wherein the monoclonal antibody is a chimeric antibody.

43. The method of claim 37, wherein the monoclonal antibody is a humanized antibody.

44. The method of claim 36, wherein the portion of the antibody comprises a complementarity determining region or variable region of a light or heavy chain.

45. The method of claim 36, wherein the portion of the antibody comprises a complementarity determining region or a variable region.

46. The method of claim 45, wherein the portion of the antibody comprises a Fab or a single chain antibody.

47. The method of claim 31, wherein the subject is a mammal.

48. The method of claim 47, wherein the mammal is a rodent.

49. The method of claim 47, wherein the mammal is a human.

50. The method of claim 31, wherein the protein comprises soluble extracellular region of CD40 ligand, or variant thereof including conservative substituents, or portion thereof; or soluble extracellular region of CD40, or variant thereof including conservative substituents, or portion thereof.

51. The method of claim 50, wherein the soluble extracellular region of CD40 ligand or CD40 is a monomer.

52. The method of claim 50, wherein the soluble extracellular region of CD40 is an oligomer.

53. The method of claim 50, wherein the protein comprising soluble extracellular region of CD40 or portion thereof or CD40 ligand or portion thereof further comprises an Fc region fused to the extracellular region of CD40 or portion thereof or CD40 ligand or portion thereof.

54. The method of claim 53, wherein the Fc region is capable of binding to protein A or protein G.

55. The method of claim 53, wherein the Fc region comprises IgG, IgA, IgM, IgD, or IgE, or subclasses thereof.

56. The method of claim 55, wherein:
the IgG is IgG₁, IgG₂, IgG₃, or IgG₄; or
the IgA is IgA₁ or IgA₂.

57. The method of claim 31, wherein the agent is nonprotein.

58. The method of claim 57, wherein the agent is a small molecule.

59. The method of claim 31, wherein the agent is selected from a library of known agents.

60. The method of claim 31, wherein the agent is modified from a known agent.

61. The method of claim 60, wherein the modified agent is designed by structure optimization of a lead inhibitor based on a three-dimensional structure of a complex of soluble extracellular region of CD40 ligand or portion thereof with the lead inhibitor.

62. The method of claim 31, wherein the agent is selected by a screening method, which comprises:
isolating a sample of cells;
culturing the sample under conditions permitting activation of CD40-bearing cells;
contacting the sample with cells expressing a protein which is specifically recognized by monoclonal antibody 5c8 produced by the hybridoma having ATCC Accession No. HB 10916, or with a protein which is specifically recognized by monoclonal antibody 5c8 produced by the hybridoma having ATCC Accession No. HB 10916, effective to activate the CD40-bearing cells;
contacting the sample with an amount of the agent effective to inhibit activation of the CD40-bearing cells if the agent is capable of inhibiting activation of the CD40-bearing cells; and
determining whether the cells expressing the protein which is specifically recognized by monoclonal antibody 5c8 produced by the hybridoma having ATCC Accession No. HB 10916, or with the protein which is specifically recognized by monoclonal antibody 5c8 produced by the hybridoma having ATCC Accession No.
HB 10916,activate the CD40-bearing cells in the presence of the agent.

63. The method of claim 62, wherein the agent is selected from a library of known agents.

64. The method of claim 63, wherein the known agents are nonprotein agents.

65. A method of treating, in a subject, a smooth muscle cell-dependent disease, comprising inhibiting activation by CD40 ligand of smooth muscle cells bearing CD40 on the surface of the cells according to the method of claim 31.

66. The method of claim 65, wherein the smooth muscle cell-dependent disease is a vascular disease.

67. The method of claim 66, wherein the vascular disease is atherosclerosis.

68. The method of claim 65, wherein the smooth muscle cell-dependent disease is a gastrointestinal disease.

69. The method of claim 68, wherein the gastrointestinal disease is selected from the group consisting of: esophageal dysmotility, inflammatory bowel disease, and scleroderma.

70. The method of claim 65, wherein the smooth muscle cell-dependent disease is a bladder disease.


SEQUENCE LISTING (1 sequence)

SEQ ID NO: 1 - protein, 146 aa
  hypothetical: NO
GDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL